(19) United States
(12) Patent Application Publication
(10) Pub. No.: US 2009/0181086 A1
(43) Pub. Date: Jul. 16, 2009

(54) Rasagiline formulations, their preparation and use

(76) Inventors: Muhammad Safadi, Nazareth (IL); Daniella Licht, Ramat Ilan (IL); Rachel Cohen, Hadera (IL)

Correspondence Address: COOPER & DUNHAM, LLP, 30 Rockefeller Plaza, 20th Floor, NEW YORK, NY, 10112, US

(21) Appl. No.: 12/319,576

(22) Filed: Jan. 9, 2009

Related U.S. Application Data
(60) Provisional application No. 61/010,860, filed on Jan. 11, 2008.

(51) Int. Cl. A61K 9/62; A61K 9/00; A61K 9/28; A61K 9/32

(52) U.S. Cl. 424/480; 424/400; 424/474; 424/482

(57) ABSTRACT

Disclosed are formulations which are designed to release rasagiline mesylate while maintaining specific pharmacokinetic properties.

The application claims benefit of U.S. Provisional Application No. 61/010,860, filed Jan. 11, 2008, the contents of which are hereby incorporated by reference.

Throughout this application various publications, published patent applications, and patents are referenced. The disclosures of these documents in their entireties are hereby incorporated by reference into the application in order to more fully describe the state of the art to which the invention pertains.

### BACKGROUND OF THE INVENTION

U.S. Pat. Nos. 5,532,415, 5,387,612, 5,453,446, 5,457,133, 5,599,991, 5,744,500, 5,891,923, 5,668,181, 5,576,353, 5,519,061, 5,786,390, 6,316,504, 6,630,514 disclose R(+)-N-propargyl-1-aminoindan (“R-PAI”), also known as rasagiline. Rasagiline has been reported to be a selective inhibitor of the B-form of the enzyme monoamine oxidase (“MAO-B”) and is useful in treating Parkinson's disease and various other conditions by inhibition of MAO-B in the brain.

U.S. Pat. No. 6,126,968 and PCT publication WO 95/11016, hereby incorporated by reference, disclose pharmaceutical compositions comprising rasagiline.

PCT publication WO 2006/014973, hereby incorporated by reference, discloses pharmaceutical compositions comprising rasagiline.

A concern in using monoamine oxidase (“MAO”) inhibitors is the risk of hypertensive crises, often called the “cheese effect.” (Simpson, G. M. and White K. “Tyramine studies and the safety of MAOI drugs.” J Clin Psychiatry. July 1984; 45 (7 pt 2): 59-91.) This effect is caused by inhibition of peripheral MAO. A high concentration of peripheral MAO is found in the stomach.

A further concern in Parkinson's disease patients is that many patients suffer from delayed gastric emptying (Pfeiffer, R. F. and Quigley, E. M. M. “Gastrointestinal motility problems in patients with Parkinson's disease: Epidemiology, pathophysiology, and guidelines for management,” CNS-Drugs, 1999, 11(6): 435-448; Jost, W. H., “Gastrointestinal motility problems in patients with Parkinson's disease: Effects of antiparkinsonian treatment and guidelines for management”, Drugs and Aging, 1997, 10(4): 249-258). Delayed gastric emptying (prolonged gastric residence) can be a cause of increased inhibition of peripheral MAO, and can contribute to the cheese effect.

AZILECT® is indicated for the treatment of the signs and symptoms of idiopathic Parkinson's disease as initial monotherapy and as adjunct therapy to levodopa. Rasagiline, the active ingredient of AZILECT®, is rapidly absorbed, reaching peak plasma concentration (Cₘₐₓ) in approximately 1 hour. The absolute bioavailability of rasagiline is about 36%. (AZILECT® Product Label, May 2006).

Food does not affect the Tₘₐₓ of rasagiline, although Cₘₐₓ and exposure (AUC) are decreased by approximately 60% and 20%, respectively, when the drug is taken with a high fat meal. Because AUC is not significantly affected, AZILECT® can be administered with or without food. (AZILECT® Product Label, May 2006).

The mean volume of distribution at steady-state is 87 L, indicating that the tissue binding of rasagiline is in excess of plasma protein binding. Plasma protein binding ranges from 88-94% with mean extent of binding of 61-63% to human albumin over the concentration range of 1-100 ng/mL. (AZILECT® Product Label, May 2006).

Rasagiline undergoes almost complete biotransformation in the liver prior to excretion. The metabolism of rasagiline proceeds through two main pathways: N-dealkylation and/or hydroxylation to yield 1-aminoindan (AI), 3-hydroxy-N-propargyl-1aminoindan (3-OH-PAI) and 3-hydroxy-1-aminoindan (3-OH-AI). In vitro experiments indicate that both routes of rasagiline metabolism are dependent on the cytochrome P450 (CYP) system, with CYP1A2 being the major isoenzyme involved in rasagiline metabolism. Glucuronide conjugation of rasagiline and its metabolites, with subsequent urinary excretion, is the major elimination pathway. (AZILECT® Product Label, May 2006).

After oral administration of 14C-labeled rasagiline, elimination occurred primarily via urine and secondarily via feces (62% of total dose in urine and 7% of total dose in feces over 7 days), with a total calculated recovery of 84% of the dose over a period of 38 days. Less than 1% of rasagiline was excreted as unchanged drug in urine. (AZILECT® Product Label, May 2006).

Rasagiline was shown to be a potent, irreversible MAO-B selective inhibitor. MAO-B inhibition results in an increase in extracellular levels of dopamine in the striatum. The elevated dopamine level and subsequent increased dopaminergic activity are likely to mediate rasagiline's beneficial effects seen in models of dopaminergic motor dysfunction. (Rasagiline mesylate. TVP-1012 for Parkinson's disease. Investigator's Brochure. Edition number 18. Teva Pharmaceuticals Ltd. September 2006.)

### SUMMARY OF THE INVENTION

The subject invention provides a pharmaceutical composition comprising a core comprising rasagiline mesylate and at least one pharmaceutically acceptable excipient; and an acid resistant pharmaceutically acceptable coating, wherein said pharmaceutical composition releases the following percentages of rasagiline mesylate when placed in a basket apparatus in 500 mL of buffered aqueous media at 37° C. at 75 revolutions per minute for 60 minutes under the following pH conditions: a) 0% in 0.1 N HCl; and b) between 0 and 20% in a phosphate buffer solution with a pH of 6.0.

The subject invention also provides a pharmaceutical composition comprising: a core comprising rasagiline mesylate and at least one pharmaceutically acceptable excipient; and an acid resistant pharmaceutically acceptable coating, wherein the pharmaceutical composition when ingested by a human subject provides an AUC value of rasagiline of 80-130% of that of the corresponding amount of rasagiline ingested as an immediate release formulation, over the same dosage regimen interval.

The subject invention also provides a pharmaceutical composition comprising: a core comprising rasagiline mesylate and at least one pharmaceutically acceptable excipient; and an acid resistant pharmaceutically acceptable coating, wherein the pharmaceutical composition when ingested by a human subject provides a Cₘₐₓ of rasagiline 80-145% of that of the corresponding amount of rasagiline ingested as an immediate release formulation, over the same dosage regimen interval.

The subject invention also provides a pharmaceutical composition comprising: a core comprising rasagiline mesylate and at least one pharmaceutically acceptable excipient; and a coating, comprising methacrylic acid-ethyl acrylate copolymer (1:1) and at least one plasticizer wherein in the coating the ratio of methacrylic acid-ethyl acrylate copolymer (1:1) to plasticizer is between 10 to 1 and 2 to 1.

The subject invention also provides a method of treating a patient suffering from Parkinson's disease comprising administering to the patient the above pharmaceutical composition.

### BRIEF DESCRIPTION OF THE FIGURES

FIG. 1: Plasma Concentrations (0-24 hours) for each clinical test subject—Test Product A—Day 1

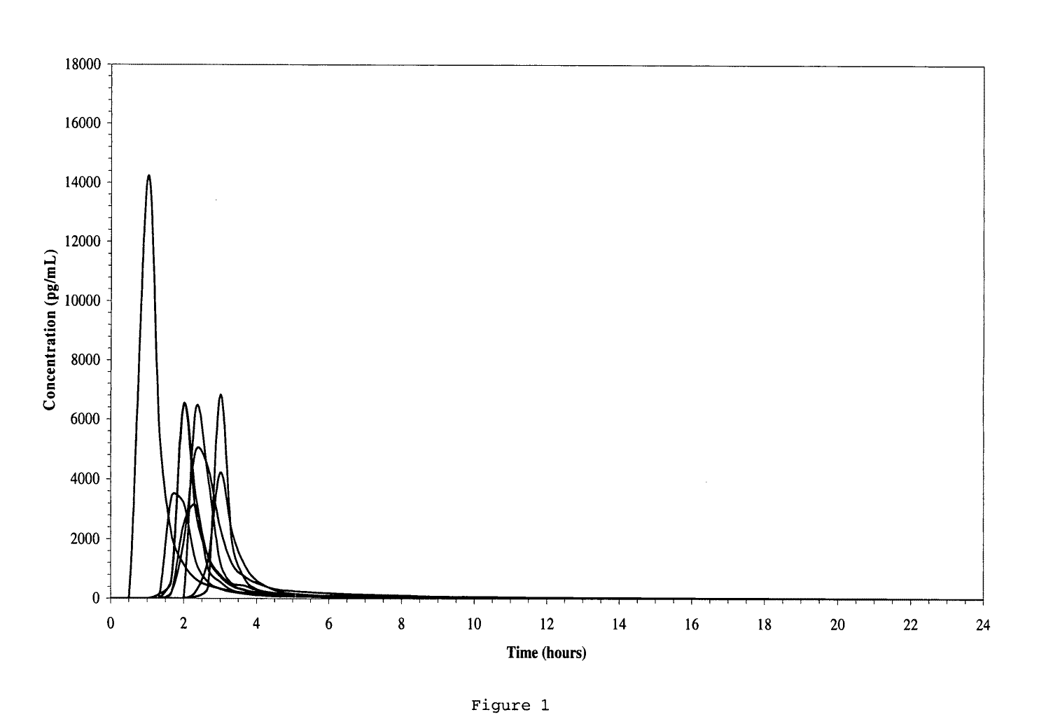

FIG. 2: Plasma Concentrations (0-36 hours) for each clinical test subject—Test Product A—Day 10

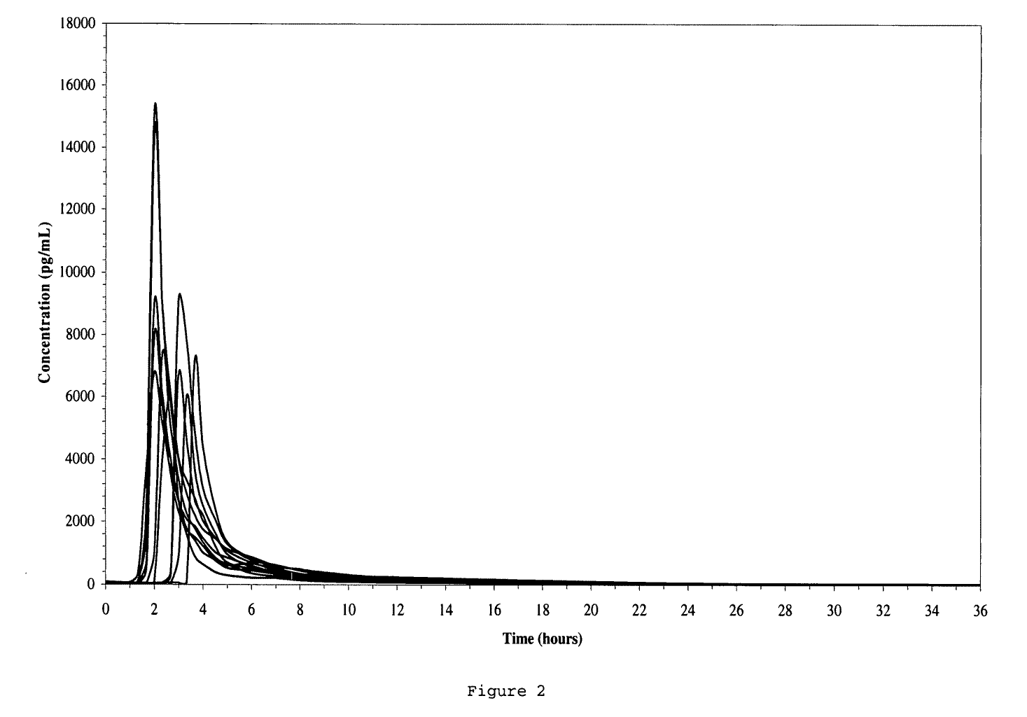

FIG. 3: Plasma Concentrations (0-24 hours) for each clinical test subject—Reference Product C—Day 1

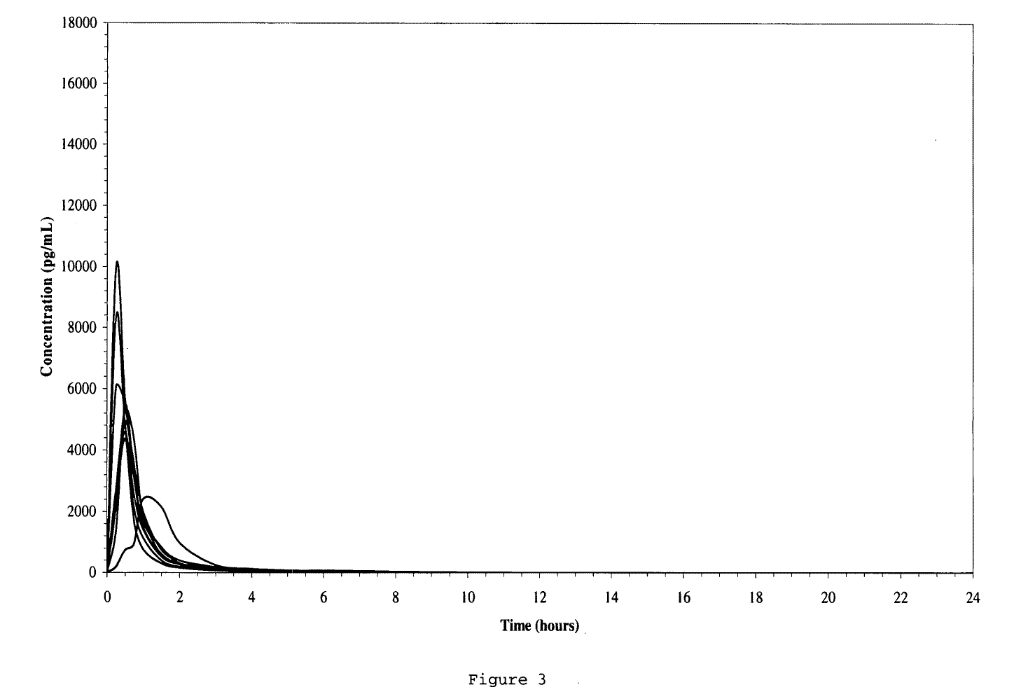

FIG. 4: Plasma Concentrations (0-36 hours) for each clinical test subject—Reference Product C—Day 10

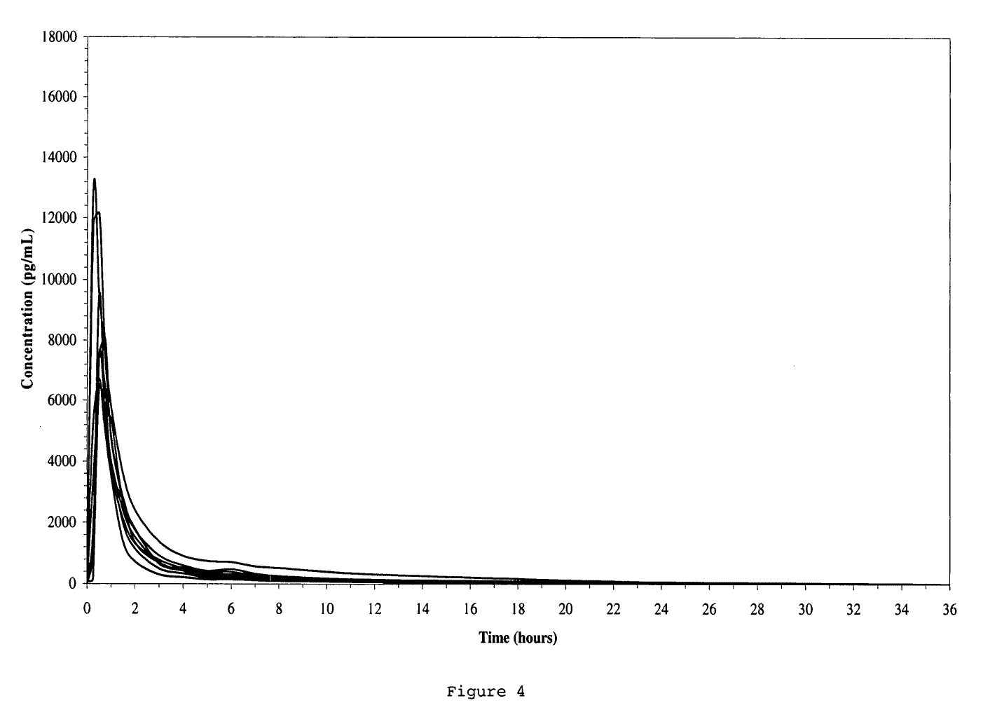

FIG. 5: Mean Plasma Concentration (0-24 hours)—Day 1

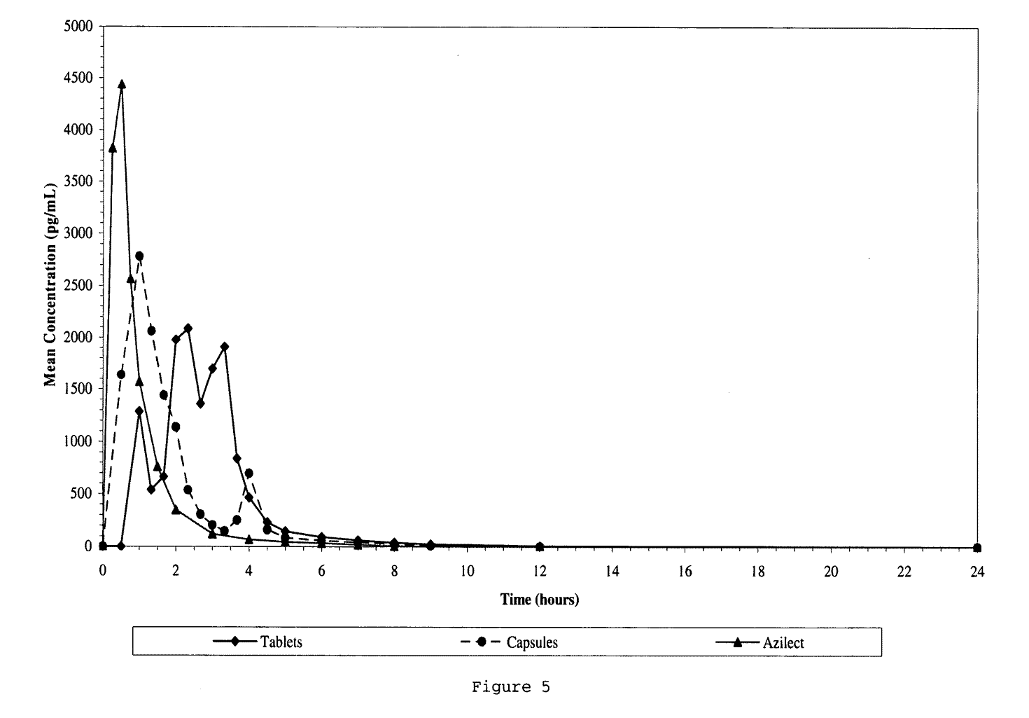

FIG. 6: Mean Plasma Concentration (0-36 hours)—Day 10

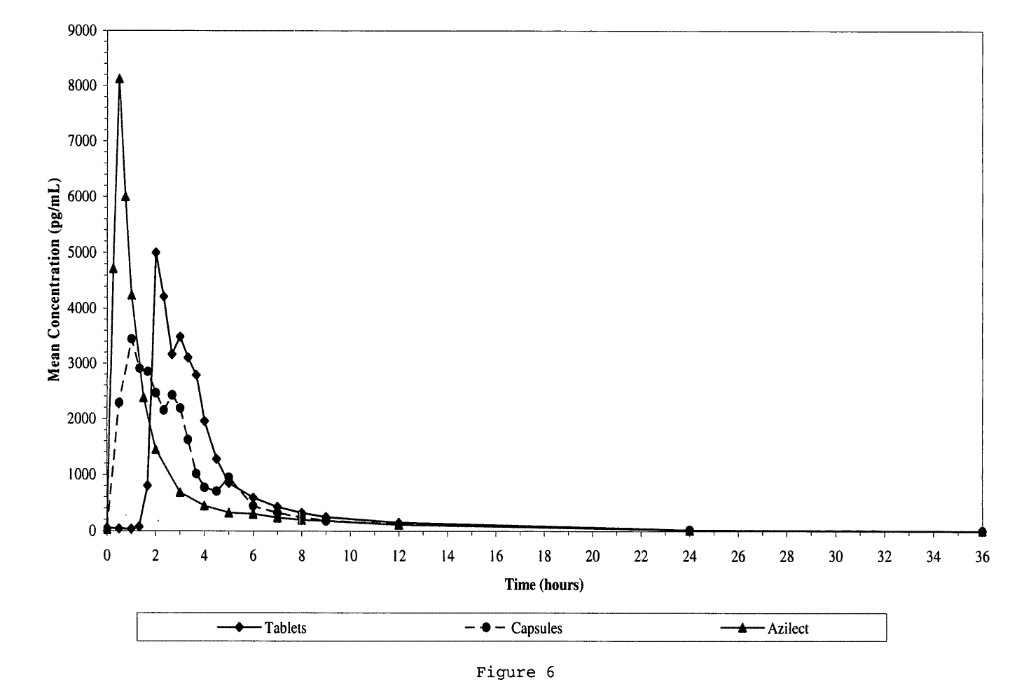

FIG. 7: Mean Plasma Concentration (0-24 hours)—Day 1—Semi-Logarithmic Scale

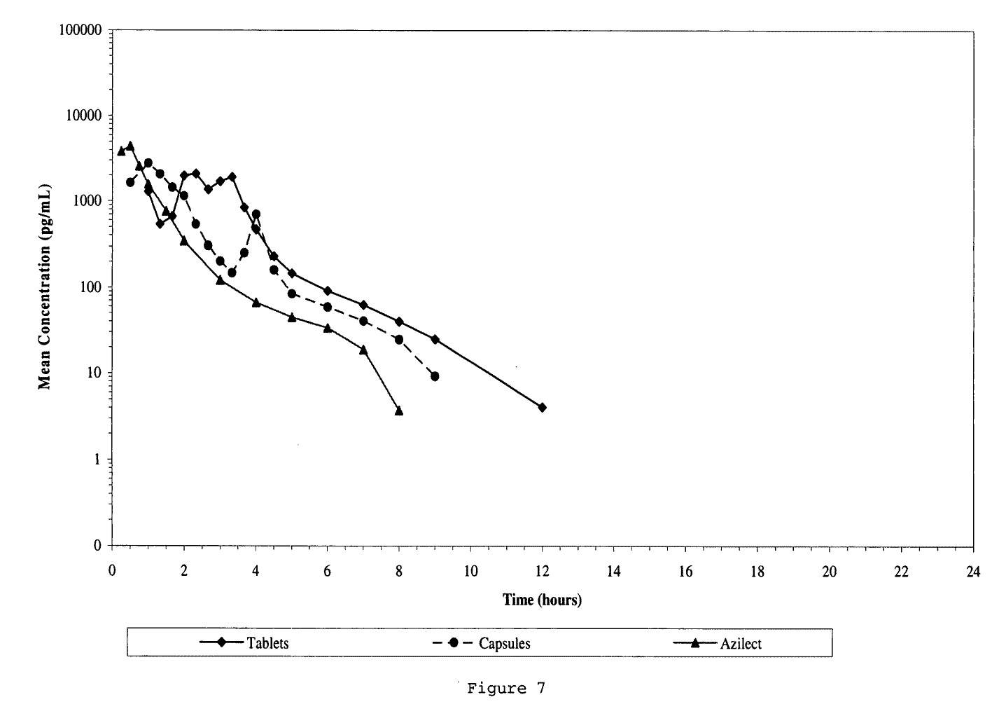

FIG. 8: Mean Plasma Concentration (0-36 hours)—Day 10—Semi-Logarithmic Scale

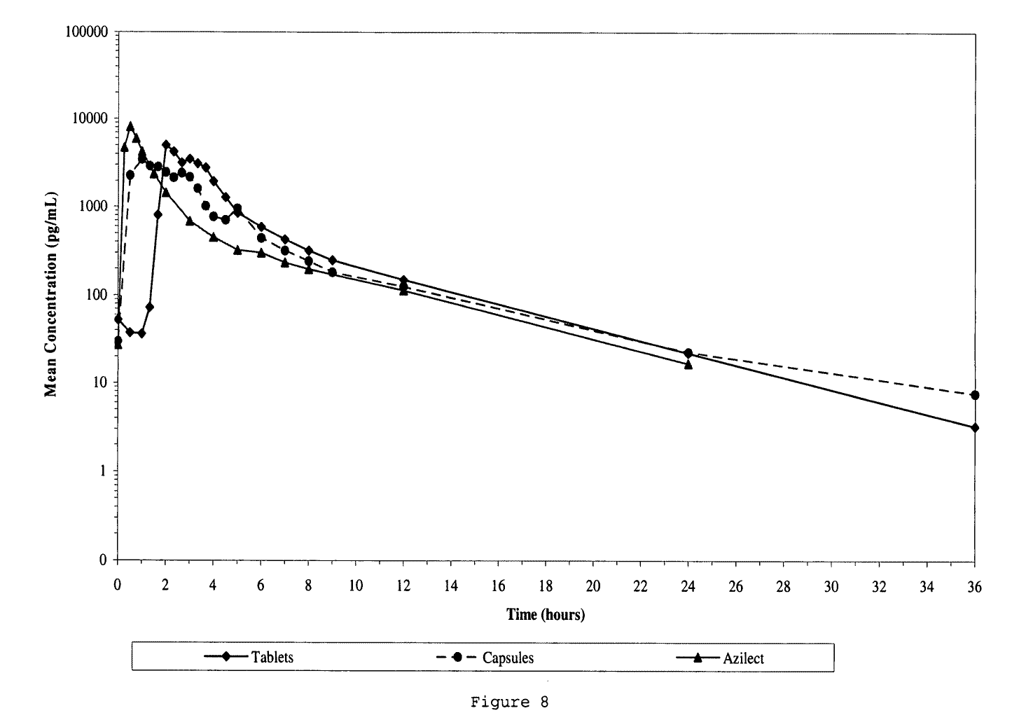

FIG. 9: Percent of MAO-B inhibition (mean±sem) by different rasagiline formulations, 6 hours post dosing on day 1 and 10.

### DETAILED DESCRIPTION OF THE INVENTION

The subject invention provides a pharmaceutical composition comprising: a core comprising rasagiline mesylate and at least one pharmaceutically acceptable excipient; and an acid resistant pharmaceutically acceptable coating wherein said pharmaceutical composition releases the following percentages of rasagiline mesylate when placed in a basket apparatus in 500 mL of buffered aqueous media at 37° C. at 75 revolutions per minute for 60 minutes under the following pH conditions: a) 0% in 0.1 N HCl; b) between 0 and 20% in a phosphate buffer solution with a pH of 6.0.

In an embodiment of the pharmaceutical composition, between 80 and 100% of rasagiline mesylate releases when placed in a basket apparatus in 500 mL of buffered aqueous media at a pH of 6.2 at 37° C. at 75 revolutions per minute for 60 minutes.

In another embodiment of the pharmaceutical composition, between 80 and 100% of rasagiline mesylate releases when placed in a basket apparatus in 500 mL of buffered aqueous media at a pH of 6.8 at 37° C. at 75 revolutions per minute for 20 minutes.

The subject invention also provides a pharmaceutical composition comprising: a core comprising rasagiline mesylate and at least one pharmaceutically acceptable excipient; and an acid resistant pharmaceutically acceptable coating, wherein the pharmaceutical composition when ingested by a human subject provides an AUC value of rasagiline of 80-130% of that of the corresponding amount of rasagiline ingested as an immediate release formulation, over the same dosage regimen interval.

In an embodiment of the pharmaceutical composition, the pharmaceutical composition upon administration to a human subject provides an AUC value of rasagiline of 80-125% of that of the corresponding amount of rasagiline ingested as an immediate released formulation, over the same dosage regimen interval.

The subject invention also provides pharmaceutical composition comprising: a core comprising rasagiline mesylate and at least one pharmaceutically acceptable excipient; and an acid resistant pharmaceutically acceptable coating, wherein the pharmaceutical composition when ingested by a human subject provides a Cₘₐₓ of rasagiline 80-145% of that of the corresponding amount of rasagiline ingested as an immediate release formulation, over the same dosage regimen interval.

In an embodiment of the pharmaceutical composition, the pharmaceutical composition when ingested by a human subject provides a Cₘₐₓ of rasagiline of 80-125% of that of the corresponding dosage of rasagiline ingested as an immediate release formulation, over the same dosage regimen interval.

In another embodiment of the pharmaceutical composition, the core is in the form of a tablet.

In yet another embodiment of the pharmaceutical composition, the core is in the form of a tablet and further comprises at least one disintegrant.

In yet another embodiment of the pharmaceutical composition, the acid resistant coating comprises between 5% and 12% by weight of the pharmaceutical composition.

In yet another embodiment of the pharmaceutical composition, the acid resistant coating comprises 8% by weight of the pharmaceutical composition.

In yet another embodiment of the pharmaceutical composition, the pharmaceutical composition is in tablet form.

In yet another embodiment of the pharmaceutical composition, the coating comprises methacrylic acid-ethyl acrylate copolymer (1:1) and a plasticizer.

In yet another embodiment of the pharmaceutical composition, the ratio of methacrylic acid-ethyl acrylate copolymer (1:1) to plasticizer in the coating is between 10 to 1 and 2 to 1.

In yet another embodiment of the pharmaceutical composition, the ratio of methacrylic acid-ethyl acrylate copolymer (1:1) to plasticizer in the coating is 5 to 1.

In yet another embodiment of the pharmaceutical composition, the plasticizer is triethyl citrate.

In yet another embodiment of the pharmaceutical composition, the coating comprises methacrylic acid-ethyl acrylate copolymer (1:1), a plasticizer and talc.

In yet another embodiment of the pharmaceutical composition, the pharmaceutical composition comprises an inner coating layer.

In yet another embodiment of the pharmaceutical composition, the pharmaceutical composition comprises an inner coating layer which comprises hypromellose.

In yet another embodiment of the pharmaceutical composition, the pharmaceutical composition has a weight of less than 150 mg.

In yet another embodiment of the pharmaceutical composition, the pharmaceutical composition comprises 1.56 mg of rasagiline mesylate.

In yet another embodiment of the pharmaceutical composition, the pharmaceutical composition comprises 0.78 mg of rasagiline mesylate.

In yet another embodiment of the pharmaceutical composition, the pharmaceutical composition comprises 1.56 mg or 0.78 mg of rasagiline mesylate, and mannitol, colloidal silicon dioxide, starch NF, pregelatinized starch, stearic acid, talc, hypromellose, methacrylic acid-ethyl acrylate copolymer, talc extra fine, and triethyl citrate.

In yet another embodiment of the pharmaceutical composition, the pharmaceutical composition consists of 79.84 mg of mannitol, 0.6 mg of colloidal silicon dioxide, 1.56 mg of rasagiline mesylate, 10.0 mg of starch NF, 20.0 mg of pregelatinized starch, 2.0 mg of stearic acid, 2.0 mg of talc, 4.8 mg of hypromellose, 6.25 mg of methacrylic acid-ethyl acrylate copolymer, 1.25 mg of triethyl citrate, and 3.1 mg of talc extra fine.

In yet another embodiment of the pharmaceutical composition, the pharmaceutical composition consists of 80.62 mg of mannitol, 0.6 mg of colloidal silicon dioxide, 0.78 mg of rasagiline mesylate, 10.0 mg of starch NF, 20.0 mg of pregelatinized starch, 2.0 mg of stearic acid, 2.0 mg of talc, 4.8 mg of hypromellose, 6.25 mg of methacrylic acid-ethyl acrylate copolymer, 1.25 mg of triethyl citrate, and 3.1 mg of talc extra fine.

The subject invention also provides a pharmaceutical composition comprising:
a) a core, comprising rasagiline mesylate and at least one pharmaceutically acceptable excipient; and
b) a coating, comprising methacrylic acid-ethyl acrylate copolymer (1:1) and at least one plasticizer wherein in the coating the ratio of methacrylic acid-ethyl acrylate copolymer (1:1) to plasticizer is between 10 to 1 and 2 to 1.

In an embodiment of the pharmaceutical composition, the ratio in the coating of methacrylic acid-ethyl acrylate copolymer (1:1) to plasticizer is 5 to 1.

In another embodiment of the pharmaceutical composition, the coating comprises between 5% and 12% by weight of the pharmaceutical composition.

In yet another embodiment of the pharmaceutical composition, the coating comprises 8% by weight of the pharmaceutical composition.

In yet another embodiment of the pharmaceutical composition, the plasticizer(s) are water soluble.

In yet another embodiment of the pharmaceutical composition, the plasticizer(s) are a combination of several water soluble plasticizers.

In yet another embodiment of the pharmaceutical composition, the plasticizer(s) are a combination of water soluble plasticizers and water insoluble plasticizers.

In yet another embodiment of the pharmaceutical composition, the plasticizer is triethyl citrate.

In yet another embodiment of the pharmaceutical composition, the coating further comprises lubricant(s).

In yet another embodiment of the pharmaceutical composition, the coating further comprises lubricant(s) which is talc extra fine.

In yet another embodiment of the pharmaceutical composition, the coating further comprises talc extra fine.

In yet another embodiment of the pharmaceutical composition, the core is in tablet form.

In yet another embodiment of the pharmaceutical composition, the core further comprises at least one disintegrant.

In yet another embodiment of the pharmaceutical composition, the core comprises between 0.5% and 20% by weight of disintegrant.

In yet another embodiment of the pharmaceutical composition, the core comprises between 0.5% and 20% by weight of disintegrant which comprises pre-gelatinized starch.

In yet another embodiment of the pharmaceutical composition, the pharmaceutical composition has a weight of less than 150 mg.

In yet another embodiment of the pharmaceutical composition, the pharmaceutical composition comprises 1.56 mg of rasagiline mesylate.

In yet another embodiment of the pharmaceutical composition, the pharmaceutical composition comprises 1.56 mg of rasagiline.

In yet another embodiment of the pharmaceutical composition, the pharmaceutical composition comprises 0.78 mg of rasagiline.

In yet another embodiment of the pharmaceutical composition, the pharmaceutical composition further comprises mannitol, colloidal silicon dioxide, starch NF, pregelatinized starch, stearic acid, talc, hypromellose, methacrylic acid-ethyl acrylate copolymer, talc extra fine, and triethyl citrate.

In yet another embodiment of the pharmaceutical composition, the pharmaceutical composition consists of 79.84 mg of mannitol, 0.6 mg of colloidal silicon dioxide, 1.56 mg of rasagiline mesylate, 10.0 mg of starch NF, 20.0 mg of pregelatinized starch, 2.0 mg of stearic acid, 2.0 mg of talc, 4.8 mg of hypromellose, 6.25 mg of methacrylic acid-ethyl acrylate copolymer, 1.25 mg of triethyl citrate, and 3.1 mg of talc extra fine.

In yet another embodiment of the pharmaceutical composition, the pharmaceutical composition consists of 80.62 mg of mannitol, 0.6 mg of colloidal silicon dioxide, 0.78 mg of rasagiline mesylate, 10.0 mg of starch NF, 20.0 mg of pregelatinized starch, 2.0 mg of stearic acid, 2.0 mg of talc, 4.8 mg of hypromellose, 6.25 mg of methacrylic acid-ethyl acrylate copolymer, 1.25 mg of triethyl citrate, and 3.1 mg of talc extra fine.

The subject invention also provides a method of treating a patient suffering from Parkinson's disease which comprises administering to the patient the above pharmaceutical composition.

In one embodiment of the method, the patient suffers from delayed gastric emptying.

The immediate release formulation of rasagiline is defined as AZILECT® Tablets contain rasagiline (as the mesylate), a propargylamine-based drug indicated for the treatment of idiopathic Parkinson's disease. It is designated chemically as: 1H-Inden-1-amine, 2,3-dihydro-N-2-propynyl-, (1R)-, methanesulfonate. Rasagiline mesylate is a white to off-white powder, freely soluble in water or ethanol and sparingly soluble in isopro-panol. Each AZILECT tablet for oral administration contains rasagiline mesylate equivalent to 0.5 mg or 1 mg of rasagiline base.

Each AZILECT tablet also contains the following inactive ingredients: mannitol, starch, pregelatinized starch, colloidal silicon dioxide, stearic acid and talc.

AZILECT is an irreversible monoamine oxidase inhibitor indicated for the treatment of idiopathic Parkinson's disease. AZILECT inhibits MAO type B, but adequate studies to establish whether rasagiline is selective for MAO type B (MAO-B) in humans have not yet been conducted.

MAO, a flavin-containing enzyme, is classified into two major molecular species, A and B, and is localized in mitochon-drial membranes throughout the body in nerve terminals, brain, liver and intestinal mucosa. MAO regulates the the metabolic degradation of catecholamines and serotonin in the CNS and peripheral tissues. MAO-B is the major form in the human brain. In ex vivo animal studies in brain, liver and intestinal tissues, rasagiline was shown to be a potent, irreversible monoamine oxidase type B (MAO-B) selective inhibitor. Rasagiline at the recommended therapeutic dose was also shown to be a potent and irreversible inhibitor of MAO-B in platelets. The selectivity of rasagiline for inhibiting only MAO-B (and not MAO-A) in humans and the sensitivity to tyramine during rasagiline treatment at any dose has not been sufficiently characterized to avoid restriction of dietary tyramine and amines contained in medications.

The precise mechanisms of action of rasagiline are unknown. One mechanism is believed to be related to its MAO-B inhibitory activity, which causes an increase in extracellular levels of dopamine in the striatum. The elevated dopamine level and subsequent increased dopaminergic activity are likely to mediate rasagiline's beneficial effects seen in models of dopaminergic motor dysfunction.

Studies in healthy subjects and in Parkinson's disease patients have shown that rasagiline inhibits platelet MAO-B irreversibly. The inhibition lasts at least 1 week after last dose. Almost 25-35% MAO-B inhibition was achieved after a single rasagiline dose of 1 mg/day and more than 55% of MAO-B inhibition was achieved after a single rasagiline dose of 2 mg/day. Over 90% inhibition was achieved 3 days after rasagiline daily closing at 2 mg/day and this inhibition level was maintained 3 days post-dose. Multiple doses of rasagiline of 0.5, 1 and 2 mg per day resulted in complete MAO-B inhibition.

Rasagiline's pharmacokinetics are linear with doses over the range of 1-10 mg. Its mean steady-state half life is 3 hours but there is no correlation of pharmacokinetics with its pharmacological effect because of its irreversible inhibition of MAO-B.

Rasagiline is rapidly absorbed, reaching peak plasma concentration (Cₘₐₓ) in approximately 1 hour. The absolute bioavailability of rasagiline is about 36%.

Food does not affect the Tₘₐₓ of rasagiline, although Cₘₐₓ and exposure (AUC) are decreased by approximately 60% and 20%, respectively, when the drug is taken with a high fat meal. Because AUC is not significantly affected, Azilect can be administered with or without food. (Physician Desk Reference, 63^{rd} Edition, 2009, p 3106).

MAO inhibitors that selectively inhibit MAO-B are largely devoid of the potential to cause the “cheese effect”. Nonetheless, the possibility exists that delayed gastric emptying of R-PAI may contribute to this phenomenon. Therefore, a main goal in developing the formulations of the current invention was to develop a delayed release, enteric coated formulation comprising rasagiline mesylate in an amount equivalent to 1 mg of rasagiline base which would release the active ingredient in the duodenum and the jejunum, past the stomach.

During the development of the formulations of the current invention, it was determined that the formulations should meet the criteria of bioequivalence to the known, immediate release rasagiline mesylate formulations (as disclosed in example 1) in a single dose bio-equivalence study in healthy subjects. These criteria include similarity of Cₘₐₓ and AUC₀₋ₜ (area under the curve) within the range of 80-125% within a 90% confidence interval between the new formulations and the known, immediate release formulations. The difference between the two formulations should be evident in bioequivalence studies as a difference in tₘₐₓ. In other words, the mean pharmacokinetic profile of the formulations of the current invention should match the mean pharmacokinetic profile of the formulations of the known immediate release formulation, with the exception of the tₘₐₓ which should be greater for the delayed release formulation than for the immediate release formulation.

The reason for attempting to match the mean Cₘₐₓ and AUC₀₋ₜ of the known immediate release formulation (i.e. to formulate a delayed release formulation that is bioequivalent) is that the efficacy of the immediate release formulation has been proven, and it is likely that the efficacy of the formulation relates to its mean Cₘₐₓ and/or AUC. (Arch Neurol. 2002; 59:1937-1943.)

In order to reach this target, development was directed toward enteric coated tablets having a quickly disintegrating core with an enteric coating which allows release of the rasagiline mesylate in a very specific range of pH. This specific pH range would prevent the formulation to release rasagiline mesylate in the stomach, and would allow the formulation to release rasagiline mesylate quickly under the physiological conditions of the intestine.

In PCT application publication WO 2006/014973, enteric-coated rasagiline mesylate pharmaceutical formulations were disclosed. In the disclosed formulations (Example 1, 2 and 4) methacrylic acid-ethyl acrylate copolymer (1:1) 30% dispersion, known as Eudragit® L-30 D-55 was used. As evident in the above-mentioned publication, these formulations were indeed delayed-release formulations as shown by their dissolution profiles and by the in-vivo data, however, the pharmacokinetic profile, in terms of mean Cₘₐₓ did not match the pharmacokinetic profile of the immediate release rasagiline mesylate formulations.

The excipient methacrylic acid-ethyl acrylate copolymer (1:1) 30% dispersion, known as Eudragit® L-30 D-55, used in the above-mentioned publication WO 2006/014973, when applied as an aqueous dispersion either on tablets or on spheres prevents dissolution of the coated composition at low acidic pH. The structure of this polymer is as follows:

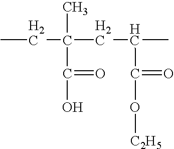

The ratio of the free carboxyl groups to the ester groups is approximately 1:1. The average molecular weight is approximately 250,000.

When this excipient is used in an aqueous dispersion or in an organic solution and formed into a film coating of a pharmaceutical formulation, it is intended to dissolve at a pH of about 5.5. (Aqueous Polymeric Coatings for Pharmaceutical Dosage Forms; Second Edition, Revised and Expanded. Ed. James W. McGinity, 1997.) It is probable that these prior art formulations began to dissolve in the stomach, perhaps in the presence of food which can raise the pH in the stomach, and continued to dissolve over a prolonged period of time in the duodenum and the jejunum. The prolonged dissolution period could explain why the Cₘₐₓ of these prior art formulations was significantly lower than the Cₘₐₓ of the immediate release formulations to which they were compared.

The compositions of the current invention are intended to withstand pH conditions of 6.0 and are intended to release the active ingredient only above that pH. This specific pH was chosen in order to avoid dissolution of the pharmaceutical compositions of the invention in the stomach and to allow rapid dissolution of the pharmaceutical compositions of the invention in the duodenum and the jejunum. The ability of a pharmaceutical formulation to enter the duodenum before releasing rasagiline mesylate and subsequently releasing the rasagiline mesylate rapidly in the duodenum provides a pharmacokinetic profile, and specifically a Cₘₐₓ and AUC₀₋ₜ, similar to that of the known immediate release formulation.

Achieving the goal of a delayed-release pharmaceutical formulation in which the Cₘₐₓ is similar to the corresponding immediate-release formulation is not trivial. In general, when delayed release formulations are compared to their immediate release counterparts in bio-studies, the Cₘₐₓ of the delayed release formulations are lower than the Cₘₐₓ in the corresponding immediate release formulations. (Mascher, et al. Arneimittelforschung. 2001; 51(6): 465-9. Behr, et al. J. Clin Pharmacol. 2002; 42(7): 791-7.)

In addition, the instant invention provides a solution to the problem of peripheral MAO inhibition by providing pharmaceutical dosage forms comprising rasagiline which are adapted to inhibit the release or absorption of rasagiline in the stomach (i.e. delay the release of rasagiline until at least a portion of the dosage form has traversed the stomach). This avoids or minimizes absorption of rasagiline in the stomach, thereby avoiding or minimizing the potential cheese effect.

The pharmaceutical dosage form may be comprised of an acid resistant excipient which prevents the dosage form or parts thereof from contacting the acidic environment of the stomach. The acid resistant excipient may coat the rasagiline in the form of an enteric coated tablet, capsule, or gelatin capsule. Enteric coating, in the context of this invention, is a coating which prevents the dissolution of an active ingredient in the stomach. This is determined by measuring the dissolution of the pharmaceutical dosage form in acidic solution, as defined by USP methods. Even in enteric pharmaceutical dosage forms, some of the dosage form may dissolve in the stomach; however, the dosage form may still be considered enteric according to USP standards.

In all of its aspects, the present invention provides an oral pharmaceutical dosage form useful for treating a condition selected from the group consisting of: Parkinson's disease, brain ischemia, head trauma injury, spinal trauma injury, neurotrauma, neurodegenerative disease, neurotoxic injury, nerve damage, dementia, Alzheimer's type dementia, senile dementia, depression, memory disorders, hyperactive syndrome, attention deficit disorder, multiple sclerosis, schizophrenia, and affective illness, but with a reduced risk of peripheral MAO inhibition that is typically associated with administration of rasagiline with known oral dosage forms.

Specific examples of pharmaceutically acceptable carriers and excipients that may be used to formulate oral dosage forms of the present invention are described, e.g., in U.S. Pat. No. 6,126,968 to Peskin et al., issued Oct. 3, 2000. Techniques and compositions for making dosage forms useful in the present invention are described, for example, in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

Tablets may contain suitable binders, lubricants, disintegrating agents, coloring agents, flavoring agents, flow-inducing agents, melting agents, and plasticizers. For instance, for oral administration in the dosage unit form of a tablet or capsule, the active drug component can be combined with an oral, non-toxic, pharmaceutically acceptable, inert carrier such as xylose, gelatin, agar, starch, methyl cellulose, dicalcium phosphate, calcium sulfate, mannitol, sorbitol, microcrystalline cellulose and the like. Suitable binders include starch, gelatin, natural sugars such as corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, stearic acid, sodium stearyl fumarate, talc and the like. Disintegrators include, without limitation, starch, methyl cellulose, agar, bentonite, xanthan gum, croscarmellose sodium, sodium starch glycolate and the like, suitable plasticizers include triacetin, triethyl citrate, dibutyl sebacate, polyethylene glycol and the like.

The basket-type apparatus used in this invention is the apparatus 1 described in the United States Pharmacopeia, 29^{th} Edition, chapter 711. The apparatus is constructed as follows:

The assembly consists of the following: a covered vessel made of glass or other inert, transparent material; a motor; a metallic drive shaft; and a cylindrical basket. The vessel is partially immersed in a suitable water bath of any convenient size or placed in a heating jacket. The water bath or heating jacket permits holding the temperature inside the vessel at 37±0.5 during the test and keeping the bath fluid in constant, smooth motion. No part of the assembly, including the environment in which the assembly is placed, contributes significant motion, agitation, or vibration beyond that due to the smoothly rotating stirring element. Apparatus that permits observation of the specimen and stirring element during the test is preferable. The vessel is cylindrical, with a hemispherical bottom and with one of the following dimensions and capacities: for a nominal capacity of 1 L, the height is 160 mm to 210 mm and its inside diameter is 98 mm to 106 mm; for a nominal capacity of 2 L, the height is 280 mm to 300 mm and its inside diameter is 98 mm to 106 mm; and for a nominal capacity of 4 L, the height is 280 mm to 300 mm and its inside diameter is 145 mm to 155 mm. Its sides are flanged at the top. A fitted cover may be used to retard evaporation. The shaft is positioned so that its axis is not more than 2 mm at any point from the vertical axis of the vessel and rotates smoothly and without significant wobble. A speed-regulating device is used that allows the shaft rotation speed to be selected and maintained at the rate specified in the individual monograph, within ±4%. Shaft and basket components of the stirring element are fabricated of stainless steel type 316 or equivalent.

Unless otherwise specified in the individual monograph, use 40-mesh cloth. A basket having a gold coating 0.0001 inch (2.5 μm) thick may be used. The dosage unit is placed in a dry basket at the beginning of each test. The distance between the inside bottom of the vessel and the basket is maintained at 25±2 mm during the test.

Within the context of this invention, dissolution is measured as an average measurement of 6 pharmaceutical dosage forms, for example, capsules or tablets.

This invention will be better understood from the experimental details which follow. However, one skilled in the art will readily appreciate that the specific methods and results discussed are merely illustrative of the invention as described more fully in the claims which follow thereafter.

### EXAMPLE 1

### Rasagiline Immediate Release Tablets

Rasagiline immediate release tablets were prepared using the ingredients listed in Table 1.

TABLE 1

| Ingredients | mg/tablet |
|---|---|
| Rasagiline mesylate | 1.56 |
| Mannitol USP | 78.84 |
| Colloidal Silicon Dioxide | 0.6 |
| Starch NF | 10.0 |
| Pregelatinized Starch NF/EP | 10.0 |
| Stearic Acid NF/EP | 2.0 |
| Talc USP/EP | 2.0 |

Rasagiline mesylate, mannitol, half of the colloidal silicon dioxide, starch and pregelatinized starch were mixed in a Diosna P-800 mixer for about 5 minutes. Water was added and the mixture was mixed further. The granulate was dried and the remainder of the colloidal silicon dioxide was added. The granulate was ground in a Frewitt mill and stearic acid and talc were added. The granulate was mixed for five minutes in a tumbler and was tableted.

### EXAMPLE 2

### Rasagiline Capsules Containing Enteric Coated Particles

Rasagiline capsules were prepared according to example 3 in PCT application publication WO 2006/014973.

These capsules were tested for dissolution in 500 ml of various aqueous acidic media made from phthalate buffer adjusted to the target pH from 2.4 to 3.6 using HCl solution and adjusted to the target pH of 4.2 to 5.2 using NaOH solution.

TABLE 2
Dissolution of capsules, in different pH media,
in percent

| Time | | | | | |
|---|---|---|---|---|---|
| (min) | pH 2.4 | pH 3.0 | pH 3.6 | pH 4.2 | pH 5.2 |
| 30 | 0 | 0 | 0 | 0 | 0 |
| 60 | 0 | 0 | 0 | 0 | 22 |
| 90 | 0 | 0 | 0 | 0 | 48 |
| 120 | 0 | 0 | 0 | 0 | 66 |

The capsule formulation begins to dissolve after 60 minutes in medium with a pH of 5.2. This may explain the lower Cₘₐₓ value in a single dose, crossover comparative pharmacokinetic study in 12 healthy male volunteers in the fasting state attributed to this formulation when compared to the immediate release formulation of example 1. It is likely that the dissolution of this formulation occurs slowly from the time the formulation enters the duodenum until the formulation proceeds in the intestine to the jejunum. Without being bound by theory, this may be attributed to the fact that the capsule disintegrates in the stomach and the coated pellets travel at different speeds through the intestine, releasing the rasagiline over a longer period of time, over a larger intestinal surface area.

### EXAMPLE 3

### Rasagiline Tablet Cores

An attempt was made to formulate tablet cores which would have a pharmacokinetic profile (Cₘₐₓ and AUC) resembling that of the immediate release formulation of example 1.

A series of tablet core formulations based on tablet formulations disclosed in U.S. Pat. No. 6,126,968 was manufactured using the amounts of excipients in Table 1.

The tablets were prepared using wet granulation technology and the amount of disintegrant was varied.

TABLE 3a
Composition of the cores of the enteric coated
tablets - All tablets included the following ingredients
in the following amounts, in mg/tablet core:

| Ingredient | Amount |
|---|---|
| Mannitol USP/EP | 159.24 |
| Colloidal Silicon Dioxide | 1.2 |
| (Aerosil ® 200) | |
| Rasagiline Mesylate | 1.56 |
| Starch NF/EP | 20.0 |
| Stearic Acid | 4.0 |
| Talc | 4.0 |

TABLE 3b
8 different formulations were prepared using the
ingredients in Table 1a while varying the excipients
below.

| Ingredient | A | B | C | D | E | F | G | H |
|---|---|---|---|---|---|---|---|---|
| Pregelatinized | 20.0 | 40.0 | 20.0 | 40.0 | 20.0 | 40.0 | 20.0 | 40.0 |
| Starch (STA- | | | | | | | | |
| RX ® 1500) | | | | | | | | |
| Croscarmellose | — | — | 5.0 | 5.0 | — | — | 5.0 | 5.0 |
| Sodium | | | | | | | | |
| (Ac-Di- | | | | | | | | |
| Sol ®, within | | | | | | | | |
| granulate) | | | | | | | | |
| Croscarmellose | — | — | — | — | 5.0 | 5.0 | 5.0 | 5.0 |
| Sodium | | | | | | | | |
| (Ac-Di- | | | | | | | | |
| Sol ®, extra | | | | | | | | |
| granular) | | | | | | | | |

The tablet cores were manufactured as follows: Mannitol, half of the colloidal silicon dioxide, rasagiline mesylate, starch NF, pre-gelatinized starch, and croscarmelose sodium (where applicable) were mixed in a high shear granulating mixer. Purified water was added, and mixing continued. The granulate was dried in a fluid bed drier and cooled to about 25° C. The remainder of the colloidal silicon dioxide was further added and the granulate was milled in an oscillating granulator with a 0.6 mm screen. Stearic acid and talc were added and the granulate was mixed in a Y-cone mixer. The granulate was then pressed into tablets.

Tablet cores manufactured using the excipients disclosed above were tested and were determined to have fast disintegration and dissolution release.

Tablet cores according to formulation B were chosen for continued development because they gave better compressibility properties and a higher hardness value compared to the other formulations, while maintaining a fast disintegration.

The dissolution percentage of tablet cores according to formulation B was tested using 0.1N HCl, paddle apparatus operated at 50 rpm, in 500 ml of dissolution media. The results are listed in table 3c.

TABLE 3c

| Time | Percent Dissolution |
|---|---|
| 5 | 0 |
| 10 | 97 |
| 15 | 97 |
| 20 | 97 |

This example shows that the dissolution of rasagiline mesylate tablet cores according to formulation B is rapid.

### EXAMPLE 4

### Rasagiline Mesylate Coated Tablets

Tablets were prepared using the tablet cores prepared according to example 3, formulation B, using the following excipients:

TABLE 4a
Enteric coated Formulation F

| | |
|---|---|
| Tablet cores B | 235.0 mg |
| Methacrylic Acid - Methyl | 14.1 mg |
| Methacrylate Copolymer | |
| [1:1] (Eudragit ® L-100) | |
| Triethyl citrate | 4.9 mg |

*this formulation can also contain talc extra-fine.

Eudragit® L-100 (Methacrylic Acid-Methyl Methacrylate Copolymer [1:1]) and triethyl citrate were added to ethanol to attain a solution. The tablets were sprayed with the solution in an Ohara coater coating pan. The inlet air temperature was between 30° C. to 40° C., the outlet air temperature was in range of 30-35° C. The pan speed was set to 7 rpm, and the spraying rate was 10-20 rpm. The nozzle diameter was 0.8 mm to 1.2 mm. The tablets were dried for 2 hours at the same conditions in the coating pan, on minimum pan speed.

The dissolution profile of the coated tablets in 0.1 N HCl was acceptable according to United States Pharmacopeia specification for delayed release (enteric coated) articles, 29^{th} edition, Chapter 724, showing less than 10% release after 120 minutes.

The dissolution profiles of the product in 500 ml of different pH media (5.4-6.8) in basket apparatus at 75 rpm at 37° C. are presented in table 4b. The media with a pH from 6.0 to 6.8 were potassium phosphate buffer media adjusted to the target pH with NaOH solution. The media with a pH from 5.4 to 5.6 were phthalate buffer media adjusted to the target pH with NaOH solution.

TABLE 4b
Dissolution results (in percent) for formulation
F in various phospate buffer media

| Time | pH 5.4 | pH 5.6 | pH 6.0 | pH 6.2 | pH 6.8 |
|---|---|---|---|---|---|
| 15 | 0 | 0 | 0 | 0 | 5 |
| 20 | 0 | 0 | 0 | 4 | 23 |
| 30 | 0 | 0 | 0 | 14 | 88 |
| 60 | 0 | 0 | 35 | 47 | 94 |
| 120 | 0 | 0 | 88 | 108 | 94 |

As evident in table 4b, there was no release at pH 5.4 or 5.6, but from pH 6.0 and above, a slow release of rasagiline was observed.

### EXAMPLE 5

### Additional Rasagiline Mesylate Coated Tablets

In order to make tablets which would not dissolve in a pH of 6.0-6.4 in a basket apparatus after 60 minutes, but would dissolve in a pH of 6.6-6.8, the amount of the water soluble plasticizer triethyl citrate was decreased to 20% of the coating while the percent of the coating layer relative to the core was increased. The excipients used for formulation G are described in table 5a.

TABLE 5a
Enteric coated Formulation G

| | |
|---|---|
| Tablet cores B | 235.0 mg |
| Methacrylic Acid - Methyl | 23.5 mg |
| Methacrylate Copolymer | |
| [1:1] (Eudragit ® L-100) | |
| Triethyl citrate | 4.7 mg |

Tablets according to formulation G were manufactured as follows. Cores were coated as in Example 4, with the exception of adjusting the amount of coating and of plasticizer.

The dissolution profile of the coated tablets in 0.1N HCl was acceptable according to United States Pharmacopeia specifications for delayed release (enteric coated) articles, 29^{th} edition, Chapter 724, showing less than 10% release after 120 minutes.

The dissolution profiles of the formulation G in different pH media (6.2-6.8) in basket apparatus at 75 rpm at 37° C. are presented in table 5a. The media were made using potassium phosphate buffer media adjusted to the target pH with NaOH solution.

TABLE 5b
Dissolution results (in percent) for formulation
G in various phosphate buffer media

| Time | pH 6.2 | pH 6.4 | pH 6.8 |
|---|---|---|---|
| 15 | 0 | 0 | No Data |
| 20 | 0 | 0 | 5 |
| 30 | 0 | 0 | 44 |
| 40 | 0 | 0 | 80 |
| 50 | 0 | 0 | 98 |
| 60 | 0 | 0 | No Data |

As is evident from table 5b, no dissolution was observed between pH 6.2-6.6 over 60 minutes. In pH 6.8 a full fast release was obtained as required.

### EXAMPLE 6

### Additional Rasagiline Mesylate Coated Tablets

Formulation G from example 5 was modified by reducing the core size. The motivation in reducing the core size was to allow for a smaller tablet which would pass into the intestine quicker, thereby reducing tablet erosion. In addition to this modification, an additional coating (pre-coat) was added to prevent any possible interaction between the rasagiline mesylate in the core and the Eudragit L polymer.

Coated tablets according to formulation H were prepared using the ingredients listed in table 6.

TABLE 6a

| Ingredient | mg/tab |
|---|---|
| Mannitol | 79.84 |
| Colloidal Silicon Dioxide | 0.6 |
| Rasagiline mesylate | 1.56 |
| Starch NF | 10.0 |
| Pregelatinized Starch (STA- | 20.0 |
| RX ® 1500) | |
| Stearic Acid | 2.0 |
| Talc | 2.0 |
| Hypromellose (Pharmacoat ® | 4.8 |
| 606G) | |
| Methacrylic Acid - Methyl | 12.58 |
| Methacrylate Copolymer | |
| [1:1] (Eudragit ® L-100) | |
| Triethyl citrate | 2.516 |

The manufacture of coated tablets according to formulation H proceeded as follows:

Mannitol USP, half of the Colloidal Silicon Dioxide, Rasagiline Mesylate, and Starch NF, and Pregelatinized starch were mixed. Water was measured were mixed and granulated with water and compressed into tablets.

Tablet cores were first coated with hypromellose (Pharmacoat® 606G) as a pre-coating, followed by Methacrylic Acid-Methyl Methacrylate Copolymer [1:1] (Eudragit® L-100) to prevent any possible interaction between the rasagiline mesylate in the core and the Eudragit L polymer.

Pharmacoat® 606G (hypromellose USP) solution was prepared using 156 g of Pharmacoat® 606G, in 1,000 g of isopropyl alcohol and 500 g of purified water.

The tablet cores were sprayed with the solution in an Ohara Coater coating pan. The inlet air temperature was between 30° C. to 40° C., the outlet air temperature was in range of 30-35° C. The pan speed was set to 7 rpm, spraying rate was 10-20 rpm. The tablets were dried for 1 hour.

Eudragit® L-100 and triethyl citrate were added to isopropyl alcohol to form a solution. The tablets were sprayed with the solution in Ohara Coater coating pan at the same conditions as the Pharmacoat® 606G intermediate coat with the exception that the drying lasted 2 hours instead of 1 hour.

The dissolution profile of the coated tablets in 0.1N HCl was acceptable according to United States Pharmacopeia specification for delayed release (enteric coated) articles, 29^{th} edition, Chapter 724, showing less than 10% release after 120 minutes.

The dissolution in pH 6.8 buffer is disclosed in table 6b.

TABLE 6b

| Time | Dissolution |
|---|---|
| 20 | 1 |
| 30 | 2 |
| 50 | 61 |
| 90 | 97 |

### EXAMPLE 7

### Rasagiline Mesylate Delayed Release Tablets

TABLE 7

| | | Percentage of total |
|---|---|---|
| Ingredient | mg/tab | weight |
| Mannitol | 79.84 | 60.8 |
| Colloidal Silicon | 0.6 | 0.457 |
| Dioxide | | |
| Rasagiline mesylate | 1.56 | 1.19 |
| Starch NF | 10.0 | 7.61 |
| Pregelatinized | 20.0 | 15.2 |
| Starch (STA-RX ® | | |
| 1500) | | |
| Stearic Acid | 2.0 | 1.52 |
| Talc | 2.0 | 1.52 |
| Hypromellose | 4.8 | 3.65 |
| (Pharmacoat ® 606G) | | |
| Methacrylic Acid | 6.250 | 4.76 |
| Ethyl Acrylate | | |
| copolymer (Eudragit ® | | |
| L 100-55) | | |
| Triethyl citrate | 1.25 | 0.951 |
| Talc USP Extra Fine | 3.1 | 2.36 |

EUDRAGIT® L 100-55 contains an anionic copolymer based on methacrylic acid and ethyl acrylate. It is also known as methacrylic acid copolymer, type C. The ratio of the free carboxyl groups to the ester groups is approx. 1:1. The average molecular weight is approx. 250,000.

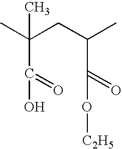

Mannitol, half of the colloidal silicon dioxide, rasagiline mesylate, starch, and pregelatinzed starch were mixed. Purified water was added to form a granulate. The granulate was dried (input temperature 55° C., output temperature 37° C.) The remainder of the colloidal silicon dioxide was added to the granulate and the granulate was milled (0.6 mm mesh.) Stearic acid and talc were than added and the granulate was then compressed into tablets.

Tablet cores were first coated with hypromellose (Pharmacoat® 606G) as a pre-coating, followed by EUDRAGIT® L 100-55 methacrylic acid and ethyl acrylate to prevent any possible interaction between the rasagiline mesylate in the core and the Eudragit L polymer.

Pharmacoat® 606G (hypromellose USP) solution was prepared using 155 g of Pharmacoat® 606G, in 1,000 g of isopropyl alcohol and 500 g of purified water.

The tablet cores were sprayed with the solution in an Ohara Coater coating pan. The inlet air temperature was between 35° C. to 40° C., the outlet air temperature was in range of 30-35° C. The pan speed was set to 8-12 rpm, spraying rate was 10-20 g/min. The tablets were dried for 2 hours.

Eudragit® L-100-55 (236.5 g) was added to 1.250 kg isopropanol, and 119 g purified water, and was mixed until a clear solution was formed. Triethyl citrate (47.3 g) in 637 g of isopropanol were added. 117.304 g of talc USP extra fine and 500 g of isopropanol were mixed together for 10 minutes, then added to the above solution. The tablets were sprayed with the solution in Ohara Coater coating pan. The inlet air temperature was between 35° C. to 38° C., the outlet air temperature was in range of 30-35° C. The pan speed was set to 14-18 rpm, spraying rate was 5-20 g/min. The tablets were dried for 2 hours.

The dissolution profile of the coated tablets in 0.1N HCl was acceptable according to United States Pharmacopeia specification for delayed release (enteric coated) articles, 29^{th} edition, Chapter 724, showing less than 10% release after 120 minutes.

### EXAMPLE 8

### Dissolution Results of Tablets According to Example 7

The tablets prepared according to example 7 from 4 different batches lettered A-D were tested for dissolution profile in various media according to USP procedures. The data below represents average for 6 tablets. The apparatus used was a Basket apparatus at 75 rpm, with 500 mL of buffered phosphate solution at various pH levels. The tablets were transferred into the buffered phosphate solution after being in a similar apparatus for 2 hours in 0.1N HCl.

TABLE 8a
% Rasagiline released - Phosphate Buffer, pH of
5.8

| Time | Batch A | Batch B | Batch C | Batch D |
|---|---|---|---|---|
| 20 | 0 | 0 | 0 | 0 |
| 30 | 0 | 0 | 0 | 0 |
| 40 | 0 | 0 | 0 | 0 |
| 50 | 0 | 0 | 0 | 0 |
| 60 | 0 | 0 | 0 | 0 |
| 70 | 0 | 0 | 0 | 0 |
| 80 | 0 | 0 | 0 | 0 |
| 90 | 0 | 0 | 0 | 1 |

TABLE 8b
% Rasagiline released - Phosphate Buffer, pH of
6.0

| Time | Batch A | Batch B | Batch C | Batch D |
|---|---|---|---|---|
| 20 | 0 | 0 | 0 | 0 |
| 30 | 0 | 0 | 0 | 0 |
| 40 | 0 | 0 | 0 | 0 |
| 50 | 0 | 0 | 0 | 0 |
| 60 | 0 | 0 | 1 | 0 |
| 70 | 0 | 0 | 5 | 2 |
| 80 | 0 | 1 | 18 | 9 |
| 90 | 0 | 2 | 35 | 24 |

TABLE 8c
% Rasagiline released - Phosphate Buffer, pH of
6.2

| Time | Batch A | Batch B | Batch C | Batch D |
|---|---|---|---|---|
| 20 | 0 | 0 | 0 | 0 |
| 30 | 0 | 2 | 20 | 13 |
| 40 | 25 | 19 | 61 | 55 |
| 50 | 86 | 64 | 84 | 87 |
| 60 | 100 | 86 | 96 | 99 |
| 70 | 100 | 93 | 96 | 99 |
| 80 | 100 | 94 | 96 | 99 |
| 90 | 100 | 94 | 96 | 100 |

TABLE 8d
% Rasagiline released - Phosphate Buffer, pH of
6.8

| Time | Batch A | Batch B | Batch C | Batch D |
|---|---|---|---|---|
| 10 | 0 | 1 | 14 | 12 |
| 20 | 106 | 91 | 97 | 92 |
| 30 | 106 | 92 | 98 | 93 |
| 40 | 106 | 93 | 99 | 94 |
| 50 | 106 | 94 | 99 | 94 |
| 70 | No Data | 95 | 99 | 94 |
| 80 | No Data | 95 | 99 | No Data |
| 90 | 106 | 95 | 99 | 94 |

### Discussion:

The tablets prepared according to Example 7 do not begin the release of rasagiline at a pH lower than 6.0. At a pH of 6.8, there is a rapid release of rasagiline and within 20 minutes, above 90% of the rasagiline is released from the formulation.

During the development of the formulations of the current invention, it was determined that the formulations should meet the criteria of bioequivalence to the known, immediate release rasagiline mesylate formulations (as disclosed in example 1) in a single dose bio-equivalence study in healthy subjects. These criteria include similarity of Cₘₐₓ and/or AUC₀₋ₜ (area under the curve) within the range of 80-125% within a 90% confidence interval between the new formulations and the known, immediate release formulations. The difference between the two formulations should be evident in bioequivalence studies as a difference in tₘₐₓ. In other words, the mean pharmacokinetic profile of the formulations of the current invention should match the mean pharmacokinetic profile of the formulations of the known immediate release formulation, with the exception of the tₘₐₓ which should be greater for the delayed release formulation than for the immediate release formulation.

The reason for attempting to match the mean Cₘₐₓ and AUC₀₋ₜ of the known immediate release formulation (i.e. to formulate a delayed release formulation that is bioequivalent) is that the efficacy of the immediate release formulation has been proven, and it is likely that the efficacy of the formulation relates to its mean Cₘₐₓ and/or AUC. (Arch Neurol. 2002; 59:1937-1943.)

In order to reach this target, development was directed toward enteric coated tablets having a quickly disintegrating core with an enteric coating which allows release of the rasagiline mesylate in a very specific range of pH. This specific pH range would prevent the formulation to release rasagiline mesylate in the stomach, and would allow the formulation to release rasagiline mesylate quickly under the physiological conditions of the intestine.

Although the tablets of example 7 were coated with an enteric coating comprising Methacrylic Acid Ethyl Acrylate copolymer, as were the compositions in PCT application publication WO 2006/014973, the tablets according to example 7 were capable of withstanding pH of 6.0 and below, whereas the composition in WO 2006/014973 were not.

The difference in dissolution profiles stems from the fact that a lower ratio of polymer to plasticizer is used in the compositions of the invention. The ratio of between 10:1 and 2:1, and specifically 5:1 allows for enhanced in vitro dissolution profiles.

The dissolution profile of the formulation of Example 7 allows the composition to have enhanced pharmacokinetic properties, similar to the currently marketed immediate release formulations.

### EXAMPLE 9

### Rasagiline Mesylate Delayed Release Tablets Prepared Using Water Only as Solvent

As detailed above, the preparation of the coating suspension in Example 7 emplyed isopropanol as a solvent. Additional formulations according to Example 7 have been prepared without using isopropanol, i.e. “water formulation.” Rasagiline mesylate enteric coated formulation Batch X and Batch Y are examples of such “water formulation”.

TABLE 9a
Batch X

| | | Reference to | | |
|---|---|---|---|---|
| Component | Function | Quality Standard | Per Tablet (mg) | |
| Core tablets | | | | |
| Rasagiline | Drug Substance | In house | 1.56* | |
| Mesylate | | standard | | |
| Mannitol | Filler | USP, BP, Ph.Eur. | 79.84 | |
| Aerosil | Flowing Agent | USP/NF | 0.6 | |
| Starch, | Disintegrant | NF, Ph.Eur. | 20.0 | |
| Pregelatinized | | | | |
| (Starch STA-RX | | | | |
| 1500) | | | | |
| Starch NF | Binder | USP, BP, Ph.Eur. | 10.0 | |
| Talc | Lubricant | USP, Ph.Eur. | 2.0 | |
| Stearic Acid | Lubricant | USP, Ph.Eur. | 2.0 | |
| Total core Tablet | | | 116.0 | |
| Weight | | | | |
| Supcoating | | | | |
| Suspention | | | | |
| Pharmacoat | Coating Agent | | 4.8 | mg |
| 606G(Hypromellose | | | | |
| USP) Granules | | | | |
| Purified Water | Processing | USP/Ph.Eur./Jp | | |
| | Agent | | | |
| Coating | | | | |
| Suspention | | | | |
| Eudragit L-30 D-55 | Coating Agent | | 6.25** | mg |
| Talc USP Extra | Lubricant | USP, Ph.Eur. | 3.1 | mg |
| Fine | | | | |
| Triethyl citrate | Plasticizer | | 1.25 | mg |
| NF | | | | |
| Purified Water | | USP/Ph.Eur./Jp | | |
| Theoretical Batch | | | | |
| Size | | | | |

*Equivalent to 1.0 mg of Rasagiline (N-propargyl-1(R)-Aminoindan Base)
**Solids remaining on the tablets

TABLE 9b
Batch Y

| | | Reference to | | |
|---|---|---|---|---|
| Component | Function | Quality Standard | Per Tablet (mg) | |
| Core tablets | | | | |
| Rasagiline | Drug Substance | In house | 1.56* | |
| Mesylate | | standard | | |
| Mannitol | Filler | USP, BP, Ph.Eur. | 79.84 | |
| Aerosil | Flowing Agent | USP/NF | 0.6 | |
| Starch, | Disintegrant | NF, Ph.Eur. | 20.0 | |
| Pregelatinized | | | | |
| (Starch STA-RX | | | | |
| 1500) | | | | |
| Starch NF | Binder | USP, BP, Ph.Eur. | 10.0 | |
| Talc | Lubricant | USP, Ph.Eur. | 2.0 | |
| Stearic Acid | Lubricant | USP, Ph.Eur. | 2.0 | |
| Total core | | | 116.0 | |
| Tablet Weight | | | | |
| Supcoating | | | | |
| Suspention | | | | |
| Pharmacoat 606G | Coating Agent | | 4.8 | mg |
| (Hypromellose | | | | |
| USP) Granules | | | | |
| Purified Water | Processing Agent | USP/Ph.Eur./Jp | | |
| Coating | | | | |
| Suspention | | | | |
| Eudragit L-30 D- | Coating Agent | | 6.25** | mg |
| 55 | | | | |
| Talc USP Extra | Lubricant | USP, Ph.Eur. | 3.1 | mg |
| Fine | | | | |
| Triethyl citrate | Plasticizer | | 1.25 | mg |
| NF | | | | |
| Purified Water | | USP/Ph.Eur./Jp | | |
| Theoretical | | | | |
| Batch Size | | | | |

Dissolution Results with Batches X and Y

The dissolution profile of the coated tablets in 0.1N HCl was acceptable according to USP specification for delayed release (enteric coated) articles, 29th edition, Chapter 724, showing less than 10% release after 120 minutes.

The dissolution profiles of the product in 500 ml of different pH media (6.0-6.8) in basket apparatus at 75 rpm at 37° C. are presented in the tables below, The media with pH from 6.0 to 6.8 were potassium phosphate buffer media adjusted to the target pH with NaOH solution,

TABLE 9c
% Rasagiline released - Phosphate buffer pH 5.8.

| | | 10 min | 20 min | 30 min | 40 min | 60 min | 90 min |
|---|---|---|---|---|---|---|---|
| Batch Y | Mean | 0 | 0 | | 0 | 0 | 0 |
| Batch X | Mean | 0 | 0 | | 0 | 0 | 0 |

TABLE 9d
% Rasagiline released - Phosphate buffer pH 6.8.

| | | 10 min | 20 min | 30 min | 40 min | 60 min | 90 min |
|---|---|---|---|---|---|---|---|
| Batch Y | Mean | 3 | 95 | 98 | 99 | 99 | 99 |
| Batch X | Mean | 2 | 85 | 89 | 89 | 90 | 90 |

These dissolution results of the “water formulation” correlate well with the dissolution results in Example 8.

### EXAMPLE 10

### Clinical Study Based on Tablets According to Example 7

This study assessed the relative bioavailability and the extent of peripheral MAO-B inhibition of Rasagiline Delayed Release Tablets (1 mg Rasagiline base) and Rasagiline Mesylate EC SGC (1 mg Rasagiline base) compared to that of AZILECT® Tablets following an oral dose once daily for 10 consecutive days (1×1 mg tablet or 1×1 mg capsule) in healthy adult subjects.

### 1. Study Design

This study was an open-label, randomized, multiple-dose, three-period, three-sequence, comparative crossover study. The total duration of the study, screening through study exit, is approximately 12 weeks with at least a 21 day washout between periods. At study check-in, the subjects reported to the clinical site at least 10.5 hours prior to Day 1 and Day 10 dosing and were required to stay for 24 hours after Day 1 and Day 10 dosing. Subjects were required to comply with an at home dosing portion of the study and report to the clinical site on three separate occasions each study period to complete study related activities.

### 2. Subject Selection

A total of twelve healthy male and female subjects (4 per sequence) were selected 18-55 years of age. Sufficient numbers of subjects were screened to enroll twelve subjects. Subjects are selected from non-institutionalized subjects consisting of members of the community at large. The subjects maintained a low-tyramine diet during the study.

### 3. Study Products and Randomization

### Test Product (A)

1 tablet of test product prepared according to Example 7 with approximately 240 mL (8 fluid ounces) of room temperature water [Rasagiline Delayed Release Tablets (1 mg Rasagiline base) by Teva Pharmaceutical Industries Ltd.]

### Test Product (B)

1 capsule of test product (B) [Rasagiline Mesylate Enteric-Coated Soft Gelatin Capsules (1 mg Rasagiline base)] with approximately 240 mL (8 fluid ounces) of room temperature water once in the morning on study Days 1 through 10

### Reference Product (C)

1 tablet of reference product with approximately 240 mL (8 fluid ounces) of room temperature water [AZILECT® Tablets (1 mg Rasagiline base) by Teva Pharmaceutical Industries Ltd.; marketed by Teva Neuroscience, Inc.]

### Randomization Sequence

Sequence 1=A B C
Sequence 2=B C A
Sequence 3=C A B

Dose administration on study Days 1 and 10 occurred after an overnight fast of at least 10 hours.

Both test products are enteric-coated, delayed release formulations of rasagiline containing 1 mg rasagiline base (as the mesylate). The terms “enteric-coated (EC)” and “delayed release (DR)” are interchangeable for the purposes of this study. The abbreviation SGC is used to indicate soft gelatin capsules for the purposes of this study.

Safety assessment of subjects during study was performed as needed.

### 4. Sample Collection and Handling Procedures

Pharmacokinetic sampling (depending on randomization) occurred on the following days at the corresponding timepoints:
a) Test Products A and B:
   Day 1 within 90 minutes prior to dosing (0 hour) and after dose administration at 0.5, 1, 1.33, 1.67, 2, 2.33, 2.67, 3, 3.33, 3.67, 4, 4.5, 5, 6, 7, 8, 9, 12, and 24 hours
   Day 8 and Day 9 prior to dosing (0 hour)
   Day 10 prior to dosing (0 hour) and after dose administration at 0.5, 1, 1.33, 1.67, 2, 2.33, 2.67, 3, 3.33, 3.67, 4, 4.5, 5, 6, 7, 8, 9, 12, 24, and 36 hours
b) Reference Product C:
   Day 1 within 90 minutes prior to dosing (0 hour) and after dose administration at 0.25, 0.5, 0.75, 1, 1.5, 2, 3, 4, 5, 6, 7, 8, 12, and 24 hours
   Day 8 and Day 9 prior to dosing (0 hour)
   Day 10 prior to dosing (0 hour) and after dose administration at 0.25, 0.5, 0.75, 1, 1.5, 2, 3, 4, 5, 6, 7, 8, 12, 24, and 36 hours

A total of 76 blood samples [43 for Test Product A and Test Product B and 33 for Reference Product C] were collected for pharmacokinetic sampling.

### Pharmacodynamic Sample Collection Schedule

Day 1 within 90 minutes prior to dosing (0 hour) and 6 hours after dose administration
Day 10 at 6 hours after dose administration

Three (3) blood samples per period ×2 study periods (total of 6 samples) were collected for pharmacodynamic sampling.

### 5. Sample Analyses

a) The rasagiline and aminoindan plasma concentrations was measured using a validated bioanalytical method and according to the Bioanalytical Laboratory's Standard Operating Procedures and FDA Guidelines.
b) The determination of the MAO-B activity in platelets was performed with a non-validated method in laboratories that are GLP certified and in accordance with the principles of GLP.
c) Samples from subjects who withdraw consent or were dropped from the study were not analyzed.

For every subject, the platelet MAO-B activity obtained before the start of each period was considered the control value. Platelet MAO-B activity during drug exposure was expressed as % of control. The determination of the MAO-B activity in platelets was performed according to SOPs in laboratories that are GLP certified.

Pharmacokinetic and statistical analyses were performed for rasagiline and aminoindan plasma data. Data from subject Nos. 1-12 were analyzed if the subject completed at least two periods and was dosed with the reference product in one of the periods.

Analyses were provided separately for each formulation and each administration day. Pharmacokinetic parameters for rasagiline and aminoindan plasma concentration were calculated using standard noncompartmental approaches as indicated below for the Day 1 comparison (Gibaldi M, Perrier D., Pharmacokinetics, 2nd edition, New York: Marcel Dekker Inc., 1982):

| | |
|---|---|
| AUC₀₋ₜ | Area under the concentration-time |
| | curve from time zero to the time of |
| | the last quantifiable concentration |
| | (t), calculated using the linear |
| | trapezoidal rule. |
| AUC_{0-inf} | Area under the concentration-time |
| | curve from time zero extrapolated to |
| | infinity. |
| AUC₀₋ₜ/AUC_{0-inf} | The ratio of AUC₀₋ₜ to AUC_{0-inf} (in |
| | percentage). |
| Cₘₐₓ | Maximum or peak concentration, |
| | obtained by inspection. |
| Tₘₐₓ | Time of maximum or peak concentration, |
| | obtained by inspection. |
| T_{lag} | The time prior to the time |
| | corresponding to the first measurable |
| | (non-zero) concentration. |
| Kel | Terminal elimination rate constant, |
| | estimated by linear regression on the |
| | terminal phase of the semi-logarithmic |
| | concentration versus time curve. |
| T_{1/2} | Half life of the product. |

Pharmacokinetic parameters for rasagiline and aminoindan plasma concentration were calculated using standard noncompartmental approaches as indicated below for the Day 10 comparison (Gibaldi M., Perrier D., Pharmacokinetics, 2nd edition, New York: Marcel Dekker Inc., 1982):

| | |
|---|---|
| AUC₀₋ₜ | Area under the concentration-time |
| | curve from time zero to the time of |
| | the last quantifiable concentration |
| | (t), calculated using the linear |
| | trapezoidal rule. |
| AUC_{0-τ(ss)} | The area under the concentration |
| | versus time curve over the dosing |
| | interval (τ) at steady state; |
| | calculated using the linear |
| | trapezoidal method. |
| Cₘₐₓ₍ₛₛ₎ | Maximum or peak measured plasma |
| | concentration at steady state. |
| Cₘᵢₙ₍ₛₛ₎ | Minimum or trough measured plasma |
| | concentration at steady state. |
| Cₐᵥ₍ₛₛ₎ | The average plasma concentration at |
| | steady state obtained by the |
| | calculation: AUC_{0-τ}/τ, where τ is the |
| | dosing interval. |
| Fluctuation Index | The fluctuation at steady state, |
| | calculated as: [(Cₘₐₓ₍ₛₛ₎₋Cₘᵢₙ₍ₛₛ₎)/ |
| | Cₐᵥ₍ₛₛ₎]. |
| Tₘₐₓ₍ₛₛ₎ | Time of maximum or peak measured |
| | plasma concentration at steady state, |
| | obtained by inspection. |
| T_{lag(ss)} | The time prior to the time |
| | corresponding to the first measurable |
| | (non-zero) concentration. |
| % Peak to Trough | Calculated as: |
| Fluctuation | 100 * [(Cₘₐₓ₍ₛₛ₎ − Cₘᵢₙ₍ₛₛ₎)/Cₘᵢₙ₍ₛₛ₎]. |
| Peak to Trough | Calculated as: (Cₘₐₓ₍ₛₛ₎ − Cₘᵢₙ₍ₛₛ₎). |
| Swing | |
| Kel | Terminal elimination rate constant, |
| | estimated by linear regression on the |
| | terminal phase of the semi-logarithmic |
| | concentration versus time curve. |
| T_{1/2} | Half life of the product. |

Relative Bioavailability at Day 1 is defined as: AUC_{0-inf} (test)/AUC_{0-inf} (reference)

Relative Bioavailability at Day 10 is defined as: AUC_{0-τ} (test)/AUC_{0-τ} (reference).

Plasma concentrations below the limit of quantitization (LOQ) was labeled as ‘BLQ’ in the plasma concentration data listings and set to zero, if recorded prior to the first measurable value of each period. If a concentration was BLQ post-dose and was followed by a concentration above LOQ, this value was set to ½ LOQ for descriptive statistics. Elsewhere, BLQ values were excluded from the PK analysis. Actual sampling time was used in the pharmacokinetic analysis.

No value of Kel, AUC_{0-inf} or T_{1/2} were reported for cases that do not exhibit a terminal log-linear phase in the concentration versus time profile for Day 1 or Day 10 comparison.

Other pharmacokinetic parameters are calculated if deemed necessary.

Statistical analyses were performed for rasagiline and aminoindan plasma concentration data at Day 1 and Day 10. Data from Subject Nos. 1-12 were analyzed for single dose (Day 1) analyses if the subject received a first dose of reference product and at least one test product. Data from subject Nos. 1-12 were analyzed for multiple dose (Day 10) analyses if the subject completed at least two periods and was dosed with the reference product in one of the periods.

Individual and mean MAO-B inhibition percentages were tabulated following multiple dose administration at 6 hours after the first and last dose of each treatment and summarized by N, arithmetic mean, standard deviation, and coefficient of variation (CV %).

Individual and mean plasma concentrations of rasagiline and aminoindan were tabulated following single and multiple dose administration at each scheduled time-point during each treatment and summarized by N, arithmetic mean, standard deviation, and coefficient of variation (CV %). Concentrations BLQ were taken as zero for descriptive statistics, except for values set to ½LOQ.

Graphical displays were generated for each subject and each period as measured and after log-transformation. Mean (±SD) concentration-time curves are plotted based on scheduled sampling times relative to drug intake.

Arithmetic means, standard deviations and coefficients of variation were calculated for the parameters listed above. Additionally, geometric means were calculated for AUC₀₋ₜ, AUC_{0-inf} (Day 1 only), AUC_{0-τ} and Cₘₐₓ for Day 1 and Day 10. Data from all completed periods were included in these analyses.

Analyses of variance (ANOVA) was performed separately at Day 1 on the ln-transformed pharmacokinetic parameters AUC₀₋ₜ, AUC_{0-inf} and Cₘₐₓ and Day 10 on the ln-transformed pharmacokinetic parameters AUC_{0-τ} and Cₘₐₓ. The ANOVA model included sequence, formulation and period as fixed effects and subject nested within sequence as a random effect. Sequence was tested using subject nested within sequence as the error term. A 5% level of significance was used to test the sequence effect. Each analysis of variance included calculation of least-squares means, the difference between adjusted formulation means and the standard error associated with this difference. The above statistical analyses were done using the MIXED procedure (SAS®).

Tₘₐₓ were analyzed using nonparametric analysis (the Wilcoxon Signed Rank Test).

In agreement with the two one-sided test for bioequivalence (Schuirmann D J., A comparison of the two one-sided tests procedure and the power approach for assessing the equivalence of average bioavailability, J Pharmacokinet Biopharm 1987; 15:657-80), 90% confidence intervals for the difference between the tests and reference formulation least-squares means (LSM) were calculated for the parameters AUC₀₋ₜ, AUC_{0-inf} and Cₘₐₓ using ln-transformed data for Day 1 and AUC_{0-τ} and Cₘₐₓ for Day 10. Confidence intervals for the ratio between means were calculated using back-transformation of the confidence intervals for the ln-transformed data. The confidence intervals were expressed as a percentage relative to the LSM of the reference formulation.

Ratios of means of the tests to reference were calculated using the LSM for ln-transformed AUC₀₋ₜ, AUC_{0-inf} and Cₘₐₓ (Day 1) and AUC_{0-τ} and Cₘₐₓ (Day 10). The geometric mean values were reported. Ratios of means were expressed as a percentage of the LSM for the reference formulation.

### Results

The results of the clinical trial are summarized in the summary table below.

TABLE 10a
Cₘₐₓ and AUC Result Summary Table

| | Day 1 | Day 10 |
|---|---|---|
| Cₘₐₓ % (DR vs IR) | 89.86-141.55 | 84.41-121.31 |
| AUC % (DR vs IR) | 101.55-122.54 | 91.04-126.23 |

The above Results Summary table shows that the delayed release formulation tested (Example 7) met the criteria for bioequivalence to the known immediate release formulation.

The tables which follow show the detailed results.

TABLE 10b
Summary of AUC₀₋ₜ and Ln-Transformed AUC₀₋ₜ for Test Product A vs. Reference Product C -
Day 1

| | | Test A | Reference C | Difference | Ratio | % Ratio | Logₑ Test A | Logₑ Reference | Logₑ Ratio |
|---|---|---|---|---|---|---|---|---|---|
| Subject | Sequence | (pg-hr/mL) | (pg-hr/mL) | (Test A − Ref C) | (Test A/Ref C) | (Test A/Ref C) | Ln (Test A) | Ln (Reference C) | Ln(Ratio) |
| 1 | 2 | 4963.66 | 3687.92 | 1275.74 | 1.346 | 134.59 | 8.510 | 8.213 | 0.297 |
| 2 | 1 | 3359.43 | 3832.10 | −472.67 | 0.877 | 87.67 | 8.120 | 8.251 | −0.132 |
| 3 | 3 | 4933.30 | 3981.89 | 951.41 | 1.239 | 123.89 | 8.504 | 8.290 | 0.214 |
| 4 | 1 | 3680.46 | 5143.88 | −1463.42 | 0.716 | 71.55 | 8.211 | 8.546 | −0.335 |
| 5 | 3 | 3743.52 | 3109.66 | 633.86 | 1.204 | 120.38 | 8.228 | 8.042 | 0.186 |
| 7 | 1 | 2854.08 | 3803.10 | −949.02 | 0.750 | 75.05 | 7.957 | 8.244 | −0.287 |
| 8 | 2 | 9931.50 | 6695.44 | 3236.06 | 1.483 | 148.33 | 9.203 | 8.809 | 0.394 |
| 9 | 2 | 3193.47 | 3578.48 | −385.01 | 0.892 | 89.24 | 8.069 | 8.183 | −0.114 |
| 10 | 3 | 6009.76 | 3150.55 | 2859.21 | 1.908 | 190.75 | 8.701 | 8.055 | 0.646 |
| 11 | 1 | 4427.99 | 4856.91 | −428.92 | 0.912 | 91.17 | 8.396 | 8.488 | −0.092 |
| 12 | 3 | 6450.94 | 4458.25 | 1992.69 | 1.447 | 144.70 | 8.772 | 8.403 | 0.369 |

| | | | | | | | | |
|---|---|---|---|---|---|---|---|---|
| N | 11 | 11 | 11 | 11 | 11 | 11 | 11 | 11 |
| MEAN | 4868.01 | 4208.93 | 659.08 | 1.161 | 116.12 | 8.424 | 8.320 | 0.104 |
| STDEV | 2036.31 | 1043.77 | 1561.15 | 0.37 | 36.98 | 0.37 | 0.23 | 0.31 |
| % CV | 41.83 | 24.80 | 236.87 | 31.84 | 31.84 | 4.35 | 2.73 | |
| MEDIAN | 4427.99 | 3832.10 | 633.86 | 1.204 | 120.38 | 8.396 | 8.251 | 0.186 |
| MIN | 2854.08 | 3109.66 | −1463.42 | 0.716 | 71.55 | 7.957 | 8.042 | −0.335 |
| MAX | 9931.50 | 6695.44 | 3236.06 | 1.908 | 190.75 | 9.203 | 8.809 | 0.646 |
| GEOMETRIC MEAN | 4557.33 | 4106.20 | | | | | | |

TABLE 10c
Summary of AUC_{0-inf} and Ln-Transformed AUC_{0-inf} for Test Product A vs. Reference Product C -
Day 1

| | | Test A | Reference C | Difference | Ratio | % Ratio | Logₑ Test A | Logₑ Reference | Logₑ Ratio |
|---|---|---|---|---|---|---|---|---|---|
| Subject | Sequence | (pg-hr/mL) | (pg-hr/mL) | (Test A − Ref C) | (Test A/Ref C) | (Test A/Ref C) | Ln (Test A) | Ln (Reference C) | Ln(Ratio) |
| 1 | 2 | 5064.50 | 3804.99 | 1259.51 | 1.331 | 133.10 | 8.530 | 8.244 | 0.286 |
| 2 | 1 | 3463.01 | 3916.08 | −453.07 | 0.884 | 88.43 | 8.150 | 8.273 | −0.123 |
| 3 | 3 | 5008.45 | 4021.85 | 986.60 | 1.245 | 124.53 | 8.519 | 8.299 | 0.219 |
| 4 | 1 | 3787.37 | 5299.81 | −1512.44 | 0.715 | 71.46 | 8.239 | 8.575 | −0.336 |
| 5 | 3 | 3862.20 | 3166.26 | 695.94 | 1.220 | 121.98 | 8.259 | 8.060 | 0.199 |
| 7 | 1 | 3001.03 | 3884.81 | −883.78 | 0.773 | 77.25 | 8.007 | 8.265 | −0.258 |
| 8 | 2 | 10065.17 | 6841.42 | 3223.75 | 1.471 | 147.12 | 9.217 | 8.831 | 0.386 |
| 9 | 2 | 3333.82 | 3630.29 | −296.47 | 0.918 | 91.83 | 8.112 | 8.197 | −0.085 |
| 10 | 3 | 6096.30 | 3192.10 | 2904.20 | 1.910 | 190.98 | 8.715 | 8.068 | 0.647 |
| 11 | 1 | 4559.58 | 5144.16 | −584.58 | 0.886 | 88.64 | 8.425 | 8.546 | −0.121 |
| 12 | 3 | 6645.58 | 4530.61 | 2114.97 | 1.467 | 146.68 | 8.802 | 8.419 | 0.383 |

| | | | | | | | | |
|---|---|---|---|---|---|---|---|---|
| N | 11 | 11 | 11 | 11 | 11 | 11 | 11 | 11 |
| MEAN | 4989.73 | 4312.03 | 677.69 | 1.165 | 116.55 | 8.452 | 8.343 | 0.109 |
| STDEV | 2041.13 | 1087.48 | 1581.00 | 0.37 | 36.75 | 0.36 | 0.23 | 0.31 |
| % CV | 40.91 | 25.22 | 233.29 | 31.54 | 31.54 | 4.23 | 2.78 | |
| MEDIAN | 4559.58 | 3916.08 | 695.94 | 1.220 | 121.98 | 8.425 | 8.273 | 0.199 |
| MIN | 3001.03 | 3166.26 | −1512.44 | 0.715 | 71.46 | 8.007 | 8.060 | −0.336 |
| MAX | 10065.17 | 6841.42 | 3223.75 | 1.910 | 190.98 | 9.217 | 8.831 | 0.647 |
| GEOMETRIC MEAN | 4685.61 | 4202.37 | | | | | | |

TABLE 10d
Summary of Cₘₐₓ and Ln-Transformed Cₘₐₓ for Test
Product A vs. Reference Product C - Day 1

| | | Test A | Reference C | Difference | Ratio | % Ratio | Logₑ Test A | Logₑ Reference | Logₑ Ratio |
|---|---|---|---|---|---|---|---|---|---|
| Subject | Sequence | (pg/mL) | (pg/mL) | (Test A − Ref C) | (Test A/Ref C) | (Test A/Ref C) | Ln (Test A) | Ln (Reference C) | Ln (Ratio) |
| 1 | 2 | 6530.1 | 2401.2 | 4128.9 | 2.720 | 271.95 | 8.784 | 7.784 | 1.000 |
| 2 | 1 | 3439.7 | 5448.2 | −2008.5 | 0.631 | 63.13 | 8.143 | 8.603 | −0.460 |
| 3 | 3 | 6484 | 4924.7 | 1559.3 | 1.317 | 131.66 | 8.777 | 8.502 | 0.275 |
| 4 | 1 | 6823.8 | 6061.5 | 762.3 | 1.126 | 112.58 | 8.828 | 8.710 | 0.118 |
| 5 | 3 | 4214.6 | 4358.2 | −143.6 | 0.967 | 96.71 | 8.346 | 8.380 | −0.034 |
| 7 | 1 | 3120.9 | 4588.9 | −1468 | 0.680 | 68.01 | 8.046 | 8.431 | −0.386 |
| 8 | 2 | 14157.9 | 10031.3 | 4126.6 | 1.411 | 141.14 | 9.558 | 9.213 | 0.345 |
| 9 | 2 | 4060.2 | 3859.9 | 200.3 | 1.052 | 105.19 | 8.309 | 8.258 | 0.051 |
| 10 | 3 | 9584 | 4908.7 | 4675.3 | 1.952 | 195.25 | 9.168 | 8.499 | 0.669 |
| 11 | 1 | 6353.4 | 5287.1 | 1066.3 | 1.202 | 120.17 | 8.757 | 8.573 | 0.184 |
| 12 | 3 | 4953 | 8368 | −3415 | 0.592 | 59.19 | 8.508 | 9.032 | −0.524 |

| | | | | | | | | |
|---|---|---|---|---|---|---|---|---|
| N | 11 | 11 | 11 | 11 | 11 | 11 | 11 | 11 |
| MEAN | 6338.33 | 5476.15 | 862.17 | 1.241 | 124.09 | 8.657 | 8.544 | 0.113 |
| STDEV | 3199.17 | 2102.90 | 2642.35 | 0.63 | 62.93 | 0.45 | 0.38 | 0.47 |
| % CV | 50.47 | 38.40 | 306.48 | 50.71 | 50.71 | 5.19 | 4.41 | |
| MEDIAN | 6353.4 | 4924.7 | 762.3 | 1.126 | 112.58 | 8.757 | 8.502 | 0.118 |
| MIN | 3120.9 | 2401.2 | −3415 | 0.592 | 59.19 | 8.046 | 7.784 | −0.524 |
| MAX | 14157.9 | 10031.3 | 4675.3 | 2.720 | 271.95 | 9.558 | 9.213 | 1.000 |
| GEOMETRIC MEAN | 5748.76 | 5136.56 | | | | | | |

TABLE 10e
Summary of AUC_{0-τ(ss)} and Ln-Transformed AUC_{0-τ(ss)} for Test
Product A vs. Reference Product C - Day 10

| | | Test A | Reference C | Difference | Ratio | % Ratio | Logₑ Test A | Logₑ Reference | Logₑ Ratio |
|---|---|---|---|---|---|---|---|---|---|
| Subject | Sequence | (pg-hr/mL) | (pg-hr/mL) | (Test A − Ref C) | (Test A/Ref C) | (Test A/Ref C) | Ln (Test A) | Ln (Reference C) | Ln (Ratio) |
| 1 | 2 | 13364.30 | 9716.93 | 3647.37 | 1.375 | 137.54 | 9.500 | 9.182 | 0.319 |
| 2 | 1 | 10094.92 | 13125.99 | −3031.07 | 0.769 | 76.91 | 9.220 | 9.482 | −0.263 |
| 3 | 3 | 11355.29 | 11913.21 | −557.92 | 0.953 | 95.32 | 9.337 | 9.385 | −0.048 |
| 4 | 1 | 7124.31 | 8317.57 | −1193.26 | 0.857 | 85.65 | 8.871 | 9.026 | −0.155 |
| 5 | 3 | 8283.86 | 9217.64 | −933.78 | 0.899 | 89.87 | 9.022 | 9.129 | −0.107 |
| 7 | 1 | 11551.11 | 11713.74 | −162.63 | 0.986 | 98.61 | 9.355 | 9.369 | −0.014 |
| 8 | 2 | 20828.15 | 16270.26 | 4557.89 | 1.280 | 128.01 | 9.944 | 9.697 | 0.247 |
| 9 | 2 | 10581.13 | 9923.71 | 657.42 | 1.066 | 106.62 | 9.267 | 9.203 | 0.064 |
| 10 | 3 | 19471.85 | 9759.28 | 9712.57 | 1.995 | 199.52 | 9.877 | 9.186 | 0.691 |
| 11 | 1 | 14952.17 | 22192.51 | −7240.34 | 0.674 | 67.37 | 9.613 | 10.008 | −0.395 |
| 12 | 3 | 12732.48 | 10756.26 | 1976.22 | 1.184 | 118.37 | 9.452 | 9.283 | 0.169 |

| | | | | | | | | |
|---|---|---|---|---|---|---|---|---|
| N | 11 | 11 | 11 | 11 | 11 | 11 | 11 | 11 |
| MEAN | 12758.14 | 12082.46 | 675.68 | 1.094 | 109.44 | 9.405 | 9.359 | 0.046 |
| STDEV | 4270.80 | 4009.96 | 4381.45 | 0.37 | 36.65 | 0.33 | 0.28 | 0.30 |
| % CV | 33.48 | 33.19 | 648.45 | 33.49 | 33.49 | 3.46 | 3.03 | |
| MEDIAN | 11551.11 | 10756.26 | −162.63 | 0.986 | 98.61 | 9.355 | 9.283 | −0.014 |
| MIN | 7124.31 | 8317.57 | −7240.34 | 0.674 | 67.37 | 8.871 | 9.026 | −0.395 |
| MAX | 20828.15 | 22192.51 | 9712.57 | 1.995 | 199.52 | 9.944 | 10.008 | 0.691 |
| GEOMETRIC MEAN | 12151.82 | 11603.20 | | | | | | |

TABLE 10f
Summary of Cₘₐₓ₍ₛₛ₎ and Ln-Transformed Cₘₐₓ₍ₛₛ₎ for Test Product A vs. Reference Product C -
Day 10

| | | Test A | Reference C | Difference | Ratio | % Ratio | Logₑ Test A | Logₑ Reference | Logₑ Ratio |
|---|---|---|---|---|---|---|---|---|---|
| Subject | Sequence | (pg/mL) | (pg/mL) | (Test A − Ref C) | (Test A/Ref C) | (Test A/Ref C) | Ln (Test A) | Ln (Reference C) | Ln (Ratio) |
| 1 | 2 | 6797.50 | 6391.40 | 406.1 | 1.064 | 106.35 | 8.824 | 8.763 | 0.062 |
| 2 | 1 | 6720.20 | 8041.20 | −1321 | 0.836 | 83.57 | 8.813 | 8.992 | −0.179 |
| 3 | 3 | 7213.40 | 6432.50 | 780.9 | 1.121 | 112.14 | 8.884 | 8.769 | 0.115 |
| 4 | 1 | 5975.30 | 9488.10 | −3512.8 | 0.630 | 62.98 | 8.695 | 9.158 | −0.462 |
| 5 | 3 | 6023.20 | 7552.10 | −1528.9 | 0.798 | 79.76 | 8.703 | 8.930 | −0.226 |
| 7 | 1 | 8007.20 | 6705.00 | 1302.2 | 1.194 | 119.42 | 8.988 | 8.811 | 0.177 |
| 8 | 2 | 15272.30 | 12919.70 | 2352.6 | 1.182 | 118.21 | 9.634 | 9.467 | 0.167 |
| 9 | 2 | 7385.10 | 6797.90 | 587.2 | 1.086 | 108.64 | 8.907 | 8.824 | 0.083 |
| 10 | 3 | 14616.80 | 9832.00 | 4784.8 | 1.487 | 148.67 | 9.590 | 9.193 | 0.397 |
| 11 | 1 | 9140.70 | 12161.40 | −3020.7 | 0.752 | 75.16 | 9.120 | 9.406 | −0.286 |
| 12 | 3 | 9058.10 | 7426.20 | 1631.9 | 1.220 | 121.97 | 9.111 | 8.913 | 0.199 |

| | | | | | | | | |
|---|---|---|---|---|---|---|---|---|
| N | 11 | 11 | 11 | 11 | 11 | 11 | 11 | 11 |
| MEAN | 8746.35 | 8522.50 | 223.85 | 1.034 | 103.35 | 9.025 | 9.020 | 0.004 |
| STDEV | 3241.14 | 2297.53 | 2427.00 | 0.25 | 25.23 | 0.32 | 0.25 | 0.26 |
| % CV | 37.06 | 26.96 | 1084.23 | 24.41 | 24.41 | 3.57 | 2.78 | |
| MEDIAN | 7385.1 | 7552.1 | 587.2 | 1.086 | 108.64 | 8.907 | 8.930 | 0.083 |
| MIN | 5975.3 | 6391.4 | −3512.8 | 0.630 | 62.98 | 8.695 | 8.763 | −0.462 |
| MAX | 15272.3 | 12919.7 | 4784.8 | 1.487 | 148.67 | 9.634 | 9.467 | 0.397 |
| GEOMETRIC MEAN | 8304.88 | 8270.69 | | | | | | |

TABLE 10g
Summary of AUC₀₋ₜ and Ln-Transformed AUC₀₋ₜ for Test Product B vs. Reference Product C -
Day 1

| | | Test B | Reference C | Difference | Ratio | % Ratio | Logₑ Test B | Logₑ Reference | Logₑ Ratio |
|---|---|---|---|---|---|---|---|---|---|
| Subject | Sequence | (pg-hr/mL) | (pg-hr/mL) | (Test B − Ref C) | (Test B/Ref C) | (Test B/Ref C) | Ln (Test B) | Ln (Reference C) | Ln (Ratio) |
| 1 | 2 | 3091.14 | 3687.92 | −596.78 | 0.838 | 83.82 | 8.036 | 8.213 | −0.177 |
| 2 | 1 | 4440.74 | 3832.10 | 608.64 | 1.159 | 115.88 | 8.399 | 8.251 | 0.147 |
| 3 | 3 | 6041.23 | 3981.89 | 2059.34 | 1.517 | 151.72 | 8.706 | 8.290 | 0.417 |
| 4 | 1 | 5178.14 | 5143.88 | 34.26 | 1.007 | 100.67 | 8.552 | 8.546 | 0.007 |
| 5 | 3 | 3744.25 | 3109.66 | 634.59 | 1.204 | 120.41 | 8.228 | 8.042 | 0.186 |
| 7 | 1 | 4836.63 | 3803.10 | 1033.53 | 1.272 | 127.18 | 8.484 | 8.244 | 0.240 |
| 8 | 2 | 4332.32 | 6695.44 | −2363.12 | 0.647 | 64.71 | 8.374 | 8.809 | −0.435 |
| 9 | 2 | 2594.48 | 3578.48 | −984.00 | 0.725 | 72.50 | 7.861 | 8.183 | −0.322 |
| 10 | 3 | 4616.29 | 3150.55 | 1465.74 | 1.465 | 146.52 | 8.437 | 8.055 | 0.382 |
| 11 | 1 | 5224.07 | 4856.91 | 367.16 | 1.076 | 107.56 | 8.561 | 8.488 | 0.073 |
| 12 | 3 | 5982.85 | 4458.25 | 1524.60 | 1.342 | 134.20 | 8.697 | 8.403 | 0.294 |

| | | | | | | | | |
|---|---|---|---|---|---|---|---|---|
| N | 11 | 11 | 11 | 11 | 11 | 11 | 11 | 11 |
| MEAN | 4552.92 | 4208.93 | 344.00 | 1.114 | 111.38 | 8.394 | 8.320 | 0.074 |
| STDEV | 1089.64 | 1043.77 | 1276.64 | 0.29 | 28.83 | 0.26 | 0.23 | 0.28 |
| % CV | 23.93 | 24.80 | 371.12 | 25.89 | 25.89 | 3.13 | 2.73 | |
| MEDIAN | 4616.29 | 3832.10 | 608.64 | 1.159 | 115.88 | 8.437 | 8.251 | 0.147 |
| MIN | 2594.48 | 3109.66 | −2363.12 | 0.647 | 64.71 | 7.861 | 8.042 | −0.435 |
| MAX | 6041.23 | 6695.44 | 2059.34 | 1.517 | 151.72 | 8.706 | 8.809 | 0.417 |
| GEOMETRIC MEAN | 4421.03 | 4106.20 | | | | | | |

TABLE 10h
Summary of AUC_{o-inf} and Ln-Transformed AUC_{0-inf} for Test Product B vs. Reference Product C -
Day 1

| | | Test B | Reference C | Difference | Ratio | % Ratio | Logₑ Test B | Logₑ Reference | Logₑ Ratio |
|---|---|---|---|---|---|---|---|---|---|
| Subject | Sequence | (pg-hr/mL) | (pg-hr/mL) | (Test B − Ref C) | (Test B/Ref C) | (Test B/Ref C) | Ln (Test B) | Ln (Reference C) | Ln (Ratio) |
| 1 | 2 | 3128.31 | 3804.99 | −676.68 | 0.822 | 82.22 | 8.048 | 8.244 | −0.196 |
| 2 | 1 | 4551.23 | 3916.08 | 635.15 | 1.162 | 116.22 | 8.423 | 8.273 | 0.150 |
| 3 | 3 | 6153.26 | 4021.85 | 2131.41 | 1.530 | 153.00 | 8.725 | 8.299 | 0.425 |
| 4 | 1 | 5325.84 | 5299.81 | 26.03 | 1.005 | 100.49 | 8.580 | 8.575 | 0.005 |
| 5 | 3 | 3834.21 | 3166.26 | 667.95 | 1.211 | 121.10 | 8.252 | 8.060 | 0.191 |
| 7 | 1 | 4977.09 | 3884.81 | 1092.28 | 1.281 | 128.12 | 8.513 | 8.265 | 0.248 |
| 8 | 2 | 4397.59 | 6841.42 | −2443.83 | 0.643 | 64.28 | 8.389 | 8.831 | −0.442 |
| 9 | 2 | 2627.32 | 3630.29 | −1002.97 | 0.724 | 72.37 | 7.874 | 8.197 | −0.323 |
| 10 | 3 | 4713.95 | 3192.10 | 1521.85 | 1.477 | 147.68 | 8.458 | 8.068 | 0.390 |
| 11 | 1 | 5599.71 | 5144.16 | 455.55 | 1.089 | 108.86 | 8.630 | 8.546 | 0.085 |
| 12 | 3 | 6061.19 | 4530.61 | 1530.58 | 1.338 | 133.78 | 8.710 | 8.419 | 0.291 |

| | | | | | | | | |
|---|---|---|---|---|---|---|---|---|
| N | 11 | 11 | 11 | 11 | 11 | 11 | 11 | 11 |
| MEAN | 4669.97 | 4312.03 | 357.94 | 1.116 | 111.65 | 8.418 | 8.343 | 0.075 |
| STDEV | 1134.13 | 1087.48 | 1322.09 | 0.29 | 29.43 | 0.27 | 0.23 | 0.29 |
| % CV | 24.29 | 25.22 | 369.36 | 26.36 | 26.36 | 3.19 | 2.78 | |
| MEDIAN | 4713.95 | 3916.08 | 635.15 | 1.162 | 116.22 | 8.458 | 8.273 | 0.150 |
| MIN | 2627.32 | 3166.26 | −2443.83 | 0.643 | 64.28 | 7.874 | 8.060 | −0.442 |
| MAX | 6153.26 | 6841.42 | 2131.41 | 1.530 | 153.00 | 8.725 | 8.831 | 0.425 |
| GEOMETRIC MEAN | 4529.37 | 4202.37 | | | | | | |

TABLE 10i
Summary Of Cₘₐₓ and Ln-Transformed Cₘₐₓ for Test Product B vs. Reference Product C - Day 1

| | | Test B | Reference C | Difference | Ratio | % Ratio | Logₑ Test B | Logₑ Reference | Logₑ Ratio |
|---|---|---|---|---|---|---|---|---|---|
| Subject | Sequence | (pg/mL) | (pg/mL) | (Test B − Ref C) | (Test B/Ref C) | (Test B/Ref C) | Ln (Test B) | Ln (Reference C) | Ln (Ratio) |
| 1 | 2 | 4935.3 | 2401.2 | 2534.1 | 2.055 | 205.53 | 8.504 | 7.784 | 0.720 |
| 2 | 1 | 3748.8 | 5448.2 | −1699.4 | 0.688 | 68.81 | 8.229 | 8.603 | −0.374 |
| 3 | 3 | 6989.7 | 4924.7 | 2065 | 1.419 | 141.93 | 8.852 | 8.502 | 0.350 |
| 4 | 1 | 4696.2 | 6061.5 | −1365.3 | 0.775 | 77.48 | 8.455 | 8.710 | −0.255 |
| 5 | 3 | 3155.5 | 4358.2 | −1202.7 | 0.724 | 72.40 | 8.057 | 8.380 | −0.323 |
| 7 | 1 | 6030.8 | 4588.9 | 1441.9 | 1.314 | 131.42 | 8.705 | 8.431 | 0.273 |
| 8 | 2 | 6662.1 | 10031.3 | −3369.2 | 0.664 | 66.41 | 8.804 | 9.213 | −0.409 |
| 9 | 2 | 2307.1 | 3859.9 | −1552.8 | 0.598 | 59.77 | 7.744 | 8.258 | −0.515 |
| 10 | 3 | 4547.7 | 4908.7 | −361 | 0.926 | 92.65 | 8.422 | 8.499 | −0.076 |
| 11 | 1 | 5879.1 | 5287.1 | 592 | 1.112 | 111.20 | 8.679 | 8.573 | 0.106 |
| 12 | 3 | 5632.4 | 8368 | −2735.6 | 0.673 | 67.31 | 8.636 | 9.032 | −0.396 |

| | | | | | | | | |
|---|---|---|---|---|---|---|---|---|
| N | 11 | 11 | 11 | 11 | 11 | 11 | 11 | 11 |
| MEAN | 4962.25 | 5476.15 | −513.91 | 0.995 | 99.54 | 8.462 | 8.544 | −0.082 |
| STDEV | 1464.98 | 2102.90 | 1943.84 | 0.45 | 44.85 | 0.34 | 0.38 | 0.39 |
| % CV | 29.52 | 38.40 | −378.25 | 45.06 | 45.06 | 4.00 | 4.41 | |
| MEDIAN | 4935.3 | 4924.7 | −1202.7 | 0.775 | 77.48 | 8.504 | 8.502 | −0.255 |
| MIN | 2307.1 | 2401.2 | −3369.2 | 0.598 | 59.77 | 7.744 | 7.784 | −0.515 |
| MAX | 6989.7 | 10031.3 | 2534.1 | 2.055 | 205.53 | 8.852 | 9.213 | 0.720 |
| GEOMETRIC MEAN | 4733.82 | 5136.56 | | | | | | |

TABLE 10j
Summary of AUC_{0-τ(ss)} and Ln-Transformed AUC_{0-τ(ss)} for Test
Product B vs. Reference Product C - Day 10

| | | Test B | Reference C | Difference | Ratio | % Ratio | Logₑ Test B | Logₑ Reference | Logₑ Ratio |
|---|---|---|---|---|---|---|---|---|---|
| Subject | Sequence | (pg-hr/mL) | (pg-hr/mL) | (Test B − Ref C) | (Test B/Ref C) | (Test B/Ref C) | Ln(Test B) | Ln(Reference C) | Ln(Ratio) |
| 1 | 2 | 8372.77 | 9716.93 | −1344.16 | 0.862 | 86.17 | 9.033 | 9.182 | −0.149 |
| 2 | 1 | 13853.22 | 13125.99 | 727.23 | 1.055 | 105.54 | 9.536 | 9.482 | 0.054 |
| 3 | 3 | 16083.13 | 11913.21 | 4169.92 | 1.350 | 135.00 | 9.686 | 9.385 | 0.300 |
| 4 | 1 | 11688.32 | 8317.57 | 3370.75 | 1.405 | 140.53 | 9.366 | 9.026 | 0.340 |
| 5 | 3 | 10537.91 | 9217.64 | 1320.27 | 1.143 | 114.32 | 9.263 | 9.129 | 0.134 |
| 7 | 1 | 9033.33 | 11713.74 | −2680.41 | 0.771 | 77.12 | 9.109 | 9.369 | −0.260 |
| 8 | 2 | 17851.51 | 16270.26 | 1581.25 | 1.097 | 109.72 | 9.790 | 9.697 | 0.093 |
| 9 | 2 | 5892.15 | 9923.71 | −4031.56 | 0.594 | 59.37 | 8.681 | 9.203 | −0.521 |
| 10 | 3 | 11243.47 | 9759.28 | 1484.19 | 1.152 | 115.21 | 9.328 | 9.186 | 0.142 |
| 11 | 1 | 18143.94 | 22192.51 | −4048.57 | 0.818 | 81.76 | 9.806 | 10.008 | −0.201 |
| 12 | 3 | NA | 10756.26 | NA | NA | NA | NA | 9.283 | NA |

| | | | | | | | | |
|---|---|---|---|---|---|---|---|---|
| N | 10 | 11 | 10 | 10 | 10 | 10 | 11 | 10 |
| MEAN | 12269.98 | 12082.46 | 54.89 | 1.025 | 102.47 | 9.360 | 9.359 | −0.007 |
| STDEV | 4128.98 | 4009.96 | 2931.27 | 0.26 | 25.96 | 0.36 | 0.28 | 0.27 |
| % CV | 33.65 | 33.19 | 5340.16 | 25.34 | 25.34 | 3.84 | 3.03 | |
| MEDIAN | 11465.90 | 10756.26 | 1023.75 | 1.076 | 107.63 | 9.347 | 9.283 | 0.073 |
| MIN | 5892.15 | 8317.57 | −4048.57 | 0.594 | 59.37 | 8.681 | 9.026 | −0.521 |
| MAX | 18143.94 | 22192.51 | 4169.92 | 1.405 | 140.53 | 9.806 | 10.008 | 0.340 |
| GEOMETRIC MEAN | 11611.08 | 11603.20 | | | | | | |

TABLE 10k
Summary of Cₘₐₓ₍ₛₛ₎ and Ln-Transformed Cₘₐₓ₍ₛₛ₎ for Test Product B vs. Reference Product C -
Day 10

| | | Test B | Reference C | Difference | Ratio | % Ratio | Logₑ Test B | Logₑ Reference | Logₑ Ratio |
|---|---|---|---|---|---|---|---|---|---|
| Subject | Sequence | (pg/mL) | (pg/mL) | (Test B − Ref C) | (Test B/Ref C) | (Test B/Ref C) | Ln (Test B) | Ln (Reference C) | Ln (Ratio) |
| 1 | 2 | 5801.50 | 6391.40 | −589.9 | 0.908 | 90.77 | 8.666 | 8.763 | −0.097 |
| 2 | 1 | 9487.50 | 8041.20 | 1446.3 | 1.180 | 117.99 | 9.158 | 8.992 | 0.165 |
| 3 | 3 | 6818.40 | 6432.50 | 385.9 | 1.060 | 106.00 | 8.827 | 8.769 | 0.058 |
| 4 | 1 | 12468.70 | 9488.10 | 2980.6 | 1.314 | 131.41 | 9.431 | 9.158 | 0.273 |
| 5 | 3 | 5558.90 | 7552.10 | −1993.2 | 0.736 | 73.61 | 8.623 | 8.930 | −0.306 |
| 7 | 1 | 3466.90 | 6705.00 | −3238.1 | 0.517 | 51.71 | 8.151 | 8.811 | −0.660 |
| 8 | 2 | 10837.00 | 12919.70 | −2082.7 | 0.839 | 83.88 | 9.291 | 9.467 | −0.176 |
| 9 | 2 | 4968.20 | 6797.90 | −1829.7 | 0.731 | 73.08 | 8.511 | 8.824 | −0.314 |
| 10 | 3 | 7295.00 | 9832.00 | −2537 | 0.742 | 74.20 | 8.895 | 9.193 | −0.298 |
| 11 | 1 | 11411.80 | 12161.40 | −749.6 | 0.938 | 93.84 | 9.342 | 9.406 | −0.064 |
| 12 | 3 | NA | 7426.20 | NA | NA | NA | NA | 8.913 | NA |

| | | | | | | | | |
|---|---|---|---|---|---|---|---|---|
| N | 10 | 11 | 10 | 10 | 10 | 10 | 11 | 10 |
| MEAN | 7811.39 | 8522.50 | −820.74 | 0.896 | 89.65 | 8.890 | 9.020 | −0.142 |
| STDEV | 3053.99 | 2297.53 | 1940.17 | 0.24 | 23.75 | 0.41 | 0.25 | 0.27 |
| % CV | 39.10 | 26.96 | −236.39 | 26.49 | 26.49 | 4.66 | 2.78 | |
| MEDIAN | 7056.7 | 7552.1 | −1289.65 | 0.873 | 87.33 | 8.861 | 8.930 | −0.136 |
| MIN | 3466.9 | 6391.4 | −3238.1 | 0.517 | 51.71 | 8.151 | 8.763 | −0.660 |
| MAX | 12468.7 | 12919.7 | 2980.6 | 1.314 | 131.41 | 9.431 | 9.467 | 0.273 |
| GEOMETRIC MEAN | 7255.40 | 8270.69 | | | | | | |

TABLE 10l
Summary of Statistical Analysis for Test Product A vs. Reference Product C - Day 1
Ln-Transformed Data

| | | | | | | | 90% | |
|---|---|---|---|---|---|---|---|---|
| | | | | | | | Confidence | |
| | | | | | | | Interval | |
| | | | | | | Mean | (Lower | P-values for |
| | Least Squares Mean | | Geometric Mean | | | Square | Limit, Upper | Product |
| PK Variable | A: Test | C: Reference | A: Test | C: Reference | % Ratio | Error | Limit) | Effects |
| Cₘₐₓ | 8.664 | 8.544 | 5792.26 | 5135.62 | 112.79 | 0.09363 | (89.86, | 0.3706 |
| | | | | | | | 141.55) | |
| AUC₀₋ₜ | 8.433 | 8.329 | 4598.23 | 4140.35 | 111.06 | 0.01723 | (100.75, | 0.0784 |
| | | | | | | | 122.43) | |
| AUC_{0-inf} | 8.461 | 8.352 | 4726.20 | 4236.76 | 111.55 | 0.01602 | (101.55, | 0.0588 |
| | | | | | | | 122.54) | |

Non-Transformed Data

| | Least Squares Mean | | | Mean Square | P-values for |
|---|---|---|---|---|---|
| PK Variable | A: Test | C: Reference | % Ratio | Error | Product Effects |
| Cₘₐₓ | 6428.48 | 5520.99 | 116.44 | 3156713 | 0.2484 |
| AUC₀₋ₜ | 4932.74 | 4257.08 | 115.87 | 673502 | 0.0705 |
| AUC_{0-inf} | 5053.57 | 4360.83 | 115.89 | 672392 | 0.0640 |
| Tₘₐₓ | 2.40 | 0.47 | 510.56 | 0.5557 | 0.0010 |
| T_{lag} | 1.60 | 0.01 | 15718.85 | 0.4728 | <.0001 |
| Kel | 0.3088 | 0.5628 | 54.87 | 0.0330 | 0.0043 |
| T_{1/2} | 2.30 | 1.76 | 130.51 | 0.6078 | 0.1243 |

TABLE 10m
Summary of Statistical Analysis for Test Product A vs. Reference Product C - Day 10
Ln-Transformed Data

| | | | | | | | | P- |
|---|---|---|---|---|---|---|---|---|
| | | | | | | | 90% Confidence | values |
| | | | | | | | Interval | for |
| | Least Squares Mean | | Geometric Mean | | | Mean Squares | (Lower Limit, | Product |
| PK Variable | A: Test | C: Reference | A: Test | C: Reference | % Ratio | Error | Upper Limit) | Effects |
| Cₘₐₓ₍ₛₛ₎ | 9.027 | 9.015 | 8326.63 | 8228.25 | 101.20 | 0.05919 | (84.41, 121.31) | 0.9106 |
| AUC_{0-τ(ss)} | 9.412 | 9.348 | 12231.60 | 11476.60 | 106.58 | 0.04861 | (90.43, 125.61) | 0.5092 |

Non-Transformed Data

| | Least Squares Mean | | | Mean Square | P-values for |
|---|---|---|---|---|---|
| PK Variable | A: Test | C: Reference | % Ratio | Error | Product Effects |
| AUC_{0-τ(ss)} | 12844.00 | 11998.00 | 107.05 | 8081103 | 0.4966 |
| AUC₀₋ₜ | 12497.00 | 11702.00 | 106.80 | 7550905 | 0.5084 |
| Cₘₐₓ₍ₛₛ₎ | 8779.19 | 8484.25 | 103.48 | 4026643 | 0.7358 |
| Cₘᵢₙ₍ₛₛ₎ | 16.46 | 16.36 | 100.64 | 197.61 | 0.9863 |
| Cₐᵥ₍ₛₛ₎ | 535.18 | 499.92 | 107.05 | 14030.00 | 0.4966 |
| Tₘₐₓ₍ₛₛ₎ | 2.54 | 0.55 | 461.35 | 0.6652 | 0.0010 |
| T_{lag} | 0.15 | 0.05 | 289.33 | 0.4952 | 0.7408 |
| Kel | 0.1788 | 0.1458 | 122.6322 | 0.0028 | 0.1628 |
| T_{1/2} | 4.63 | 5.00 | 92.50 | 1.8011 | 0.5232 |
| Fluctuation Index | 16.42 | 17.63 | 93.14 | 7.5961 | 0.3202 |
| % Peak to Trough Fluctuation | 28722.00 | 17014.00 | 168.81 | 185170000 | 0.3371 |
| Peak to Trough Swing | 8762.76 | 8467.88 | 103.48 | 4015879 | 0.7356 |

TABLE 10n
Summary of Statistical Analysis for Test Product B vs. Reference Product C - Day 1
Ln-Transformed Data

| | | | | | | | 90% | |
|---|---|---|---|---|---|---|---|---|
| | | | | | | | Confidence | |
| | | | | | | Mean | Interval | P-values for |
| | Least Squares Mean | | Geometric Mean | | | Square | (Lower Limit, | Product |
| PK Variable | B: Test | C: Reference | B: Test | C: Reference | % Ratio | Error | Upper Limit) | Effects |
| Cₘₐₓ | 8.454 | 8.544 | 4695.05 | 5135.62 | 91.42 | 0.09363 | (72.84, 114.74) | 0.5023 |
| AUC₀₋ₜ | 8.373 | 8.329 | 4326.49 | 4140.35 | 104.50 | 0.01723 | (94.79, 115.19) | 0.4441 |
| AUC_{0-inf} | 8.396 | 8.352 | 4429.27 | 4236.76 | 104.54 | 0.01602 | (95.17, 114.84) | 0.4229 |

Non-Transformed Data

| | Least Squares Mean | | | Mean Square | P-values for |
|---|---|---|---|---|---|
| PK Variable | B: Test | C: Reference | % Ratio | Error | Product Effects |
| Cₘₐₓ | 4955.45 | 5520.99 | 89.76 | 3156713 | 0.4668 |
| AUC₀₋ₜ | 4472.78 | 4257.08 | 105.07 | 673502 | 0.5470 |
| AUC_{0-inf} | 4584.51 | 4360.83 | 105.13 | 672392 | 0.5321 |
| Tₘₐₓ | 1.35 | 0.47 | 288.13 | 0.5557 | 0.0078 |
| T_{lag} | 0.53 | 0.01 | 5205.74 | 0.4728 | 0.0896 |
| Kel | 0.4829 | 0.5628 | 85.79 | 0.0330 | 0.3177 |
| T_{1/2} | 2.10 | 1.76 | 119.14 | 0.6078 | 0.3251 |

TABLE 10o
Summary of Statistical Analysis for Test Product B vs. Reference Product C - Day 10
Ln-Transformed Data

| | | | | | | | 90% Confidence | |
|---|---|---|---|---|---|---|---|---|
| | | | | | | | Interval | P-values for |
| | Least Squares Mean | | Geometric Mean | | | Mean Square | (Lower Limit, | Product |
| PK Variable | A: Test | C: Reference | A: Test | C: Reference | % Ratio | Error | Upper Limit) | Effects |
| Cₘₐₓ₍ₛₛ₎ | 9.027 | 9.015 | 8326.63 | 8228.25 | 101.20 | 0.05919 | (72.46, 105.24) | 0.2238 |
| AUC_{0-τ(ss)} | 9.412 | 9.348 | 12231.60 | 11476.60 | 106.58 | 0.04861 | (84.57, 118.63) | 0.9866 |

Non-Transformed Data

| | Least Squares Mean | | | Mean Square | P-values for |
|---|---|---|---|---|---|
| PK Variable | A: Test | C: Reference | % Ratio | Error | Product Effects |
| AUC_{0-τ(ss)} | 12844.00 | 11998.00 | 107.05 | 8081103 | 0.9049 |
| AUC₀₋ₜ | 12497.00 | 11702.00 | 106.80 | 7550905 | 0.8180 |
| Cₘₐₓ₍ₛₛ₎ | 8779.19 | 8484.25 | 103.48 | 4026643 | 0.3883 |
| Cₘᵢₙ₍ₛₛ₎ | 16.46 | 16.36 | 100.64 | 197.61 | 0.9582 |
| Cₐᵥ₍ₛₛ₎ | 535.18 | 499.92 | 107.05 | 14030.00 | 0.9049 |
| Tₘₐₓ₍ₛₛ₎ | 2.54 | 0.55 | 461.35 | 0.6652 | 0.0078 |
| T_{lag} | 0.15 | 0.05 | 289.33 | 0.4952 | 0.0602 |
| Kel | 0.1788 | 0.1458 | 122.63 | 0.0028 | 0.7380 |
| T_{1/2} | 4.63 | 5.00 | 92.50 | 1.8011 | 0.4192 |
| Fluctuation Index | 16.42 | 17.63 | 93.14 | 7.5961 | 0.1039 |
| % Peak to Trough Fluctuation | 28722.00 | 17014.00 | 168.81 | 185170000 | 0.8564 |
| Peak to Trough Swing | 8762.76 | 8467.88 | 103.48 | 4015879 | 0.3875 |

### MAO Assay:

The standard method was used for the enzymatic determination of MAO, IRD-MB-051: “Determination of monoamine oxidase (MAO) by an extraction method using radiolabelled substrate in various tissues”.

Briefly, fifty (50) μl of homogenate were added to 100 μl 0.1 M phosphate buffer (pH−7.4). After preincubation of 20 minutes at 37° C., 50 μl of ¹⁴C-phenylethylamine hydrochloride (10 μM final concentration) were added and incubation continued for next 20 minutes. The reaction was then stopped by addition of citric acid 2 M.

Radioactive metabolites were extracted into toluene/ethyl acetate (1:1 v/v.), a solution of 2,5-diphenyloxazole was added to a final concentration of 0.4% and the metabolite content estimated by liquid scintillation counting. Activity of rat brain homogenate served as standard (positive control) to the assay.

Protein determination was performed by the Lowrey method.

TABLE 10p
Percent of MAO-B inhibition by different
rasagiline formulations, 6 hours after single and 10 days
dosing.

| | MAO-B | | MAO-B | | MAO-B | |
|---|---|---|---|---|---|---|
| | % inhibition | | % inhibition | | % inhibition | |
| subject | DR Tablets | | EC capsules | | AZILECT | |
| number | day 1 | day 10 | day 1 | day 10 | day 1 | day 10 |
| 1 | * | * | 8 | 98 | 46 | 99 |
| 2 | 31 | 99 | 53 | 99 | * | * |
| 3 | 41 | 100 | * | * | 44 | 99 |
| 4 | 30 | 97 | 46 | 94 | * | * |
| 5 | 46 | 99 | * | * | 36 | 98 |
| 6 | ND | ND | 32 | 92 | ND | ND |
| 7 | 31 | 99 | 46 | 98 | * | * |
| 8 | * | * | 44 | 100 | 60 | 100 |
| 9 | * | * | 53 | 97 | 39 | 98 |
| 10 | 30 | 99 | * | * | 43 | 98 |
| 11 | 31 | 99 | 44 | 100 | * | * |
| 12 | 65 | 100 | * | * | 40 | 99 |
| average | 38.1 | 99.0 | 40.8 | 97.3 | 44.0 | 98.7 |
| sd | 12.4 | 0.9 | 14.8 | 2.9 | 7.8 | 0.8 |
| N | 8 | 8 | 8 | 8 | 7 | 7 |
| sem | 4.4 | 0.3 | 5.2 | 1.0 | 3.0 | 0.3 |
| % CV | 32.5 | 0.9 | 36.2 | 2.9 | 17.8 | 0.8 |

* blood withdrawal with lithium heparin (omitted from analysis)

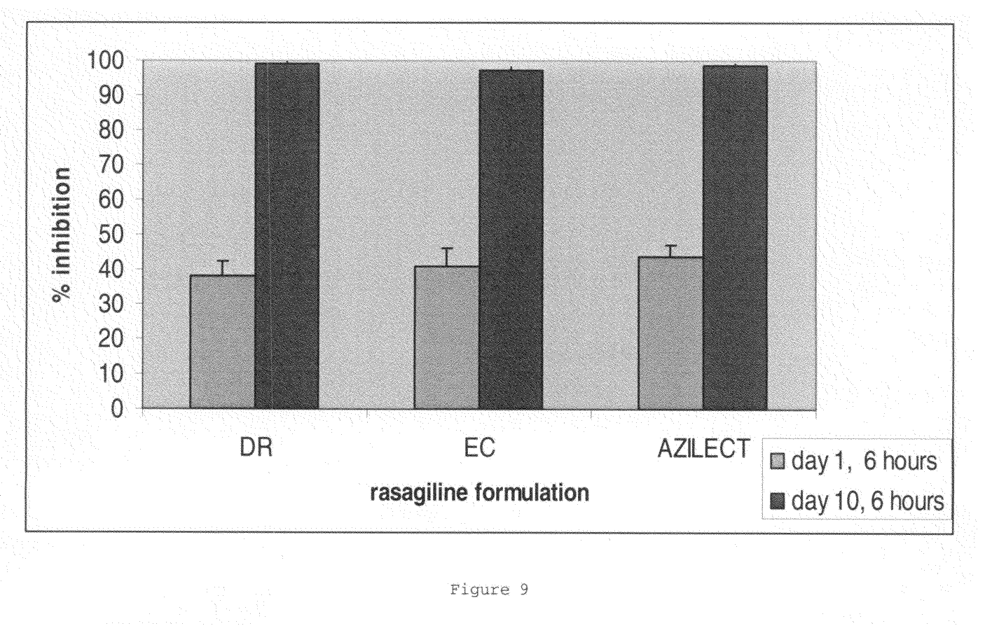

Table 10p and FIG. 9 present the percent of MAO-B inhibition compared to baseline.

After single administration, all three formulations caused abut 40% inhibition of platelets MAO-B (38% by DR tables, 41% by EC capsules and 44% by AZILECT). Full MAO-B inhibition was observed with all treatment drug administration for 10 days. Baseline activities were similar in most subjects, indicating sufficient wash out period.

1. A pharmaceutical composition comprising: a core comprising rasagiline mesylate and at least one pharmaceutically acceptable excipient; and an acid resistant pharmaceutically acceptable coating,
wherein said pharmaceutical composition releases the following percentages of rasagiline mesylate when placed in a basket apparatus in 500 mL of buffered aqueous media at 37° C. at 75 revolutions per minute for 60 minutes under the following pH conditions:
a) 0% in 0.1 N HCl;
b) between 0 and 20% in a phosphate buffer solution with a pH of 6.0.

2. The pharmaceutical composition of claim 1, which releases between 80 and 100% of rasagiline mesylate when placed in a basket apparatus in 500 mL of buffered aqueous media at a pH of 6.2 at 37° C. at 75 revolutions per minute for 60 minutes.

3. The pharmaceutical composition of claim 1, which releases between 80 and 100% of rasagiline mesylate when placed in a basket apparatus in 500 mL of buffered aqueous media at a pH of 6.8 at 37° C. at 75 revolutions per minute for 20 minutes.

4. A pharmaceutical composition comprising: a core comprising rasagiline mesylate and at least one pharmaceutically acceptable excipient; and an acid resistant pharmaceutically acceptable coating, wherein the pharmaceutical composition when ingested by a human subject provides an AUC value of rasagiline of 80-130% of that of the corresponding amount of rasagiline ingested as an immediate release formulation, over the same dosage regimen interval.

5. The pharmaceutical composition of claim 4, which upon administration to a human subject provides an AUC value of rasagiline of 80-125% of that of the corresponding amount of rasagiline ingested as an immediate released formulation, over the same dosage regimen interval.

6. A pharmaceutical composition comprising: a core comprising rasagiline mesylate and at least one pharmaceutically acceptable excipient; and an acid resistant pharmaceutically acceptable coating, wherein the pharmaceutical composition when ingested by a human subject provides a Cₘₐₓ of rasagiline 80-145% of that of the corresponding amount of rasagiline ingested as an immediate release formulation, over the same dosage regimen interval.

7. The pharmaceutical composition of claim 6, which when ingested by a human subject provides a Cₘₐₓ of rasagiline of 80-125% of that of the corresponding dosage of rasagiline ingested as an immediate release formulation, over the same dosage regimen interval.

8. The pharmaceutical composition of claim 1, wherein said core is in the form of a tablet.

9. The pharmaceutical composition of claim 1, wherein said core further comprises at least one disintegrant.

10. The pharmaceutical composition of claim 1, wherein the acid resistant coating comprises between 5% and 12% by weight of the pharmaceutical composition.

11. The pharmaceutical composition of claim 10 wherein the acid resistant coating comprises 8% by weight of the pharmaceutical composition

12. The pharmaceutical composition of claim 1, in tablet form.

13. The pharmaceutical composition of claim 1, wherein said coating comprises methacrylic acid-ethyl acrylate copolymer (1:1) and a plasticizer.

14. The pharmaceutical composition of claim 13, wherein in the coating the ratio of methacrylic acid-ethyl acrylate copolymer (1:1) to plasticizer by weight is between 10 to 1 and 2 to 1.

15. The pharmaceutical composition of claim 14, wherein in the coating the ratio of methacrylic acid-ethyl acrylate copolymer (1:1) to plasticizer by weight is 5 to 1.

16. The pharmaceutical composition of claim 13, wherein said plasticizer is triethyl citrate.

17. The pharmaceutical composition of claim 13, wherein the coating further comprises talc.

18. The pharmaceutical composition of claim 13 further comprising an inner coating layer.

19. The pharmaceutical composition of claim 18 wherein said inner coating layer comprises hypromellose.

20. The pharmaceutical composition of claim 1, having a weight of less than 150 mg.

21. The pharmaceutical composition of claim 1, comprising 1.56 mg of rasagiline mesylate.

22. The pharmaceutical composition of claim 1, comprising 0.78 mg of rasagiline mesylate.

23. The pharmaceutical composition of claim 21, further comprising mannitol, colloidal silicon dioxide, starch NF, pregelatinized starch, stearic acid, talc, hypromellose, methacrylic acid ethyl acrylate copolymer, talc extra fine, and triethyl citrate.

24. The pharmaceutical composition of claim 21, consisting of 79.84 mg of mannitol, 0.6 mg of colloidal silicon dioxide, 1.56 mg of rasagiline mesylate, 10.0 mg of starch NF, 20.0 mg of pregelatinized starch, 2.0 mg of stearic acid, 2.0 mg of talc, 4.8 mg of hypromellose, 6.25 mg of methacrylic acid-ethyl acrylate copolymer, 1.25 mg of triethyl citrate, and 3.1 mg of talc extra fine.

25. The pharmaceutical composition of claim 22, consisting of 80.62 mg of mannitol, 0.6 mg of colloidal silicon dioxide, 0.78 mg of rasagiline mesylate, 10.0 mg of starch NF, 20.0 mg of pregelatinized starch, 2.0 mg of stearic acid, 2.0 mg of talc, 4.8 mg of hypromellose, 6.25 mg of methacrylic acid-ethyl acrylate copolymer, 1.25 mg of triethyl citrate, and 3.1 mg of talc extra fine.

26. A pharmaceutical composition comprising:
a) a core comprising rasagiline mesylate and at least one pharmaceutically acceptable excipient; and
b) a coating, comprising methacrylic acid-ethyl acrylate copolymer (1:1) and at least one plasticizer, wherein in the coating the ratio of methacrylic acid-ethyl acrylate copolymer (1:1) to plasticizer by weight is between 10 to 1 and 2 to 1.

27. The pharmaceutical composition of claim 26, wherein in the coating the ratio of methacrylic acid-ethyl acrylate copolymer (1:1) to plasticizer is 5 to 1.

28. The pharmaceutical composition of claim 26, wherein the coating comprises between 5% and 12% by weight of the pharmaceutical composition.

29. The pharmaceutical composition of claim 28 wherein the coating comprises 8% by weight of the pharmaceutical composition.

30. The pharmaceutical composition of claims 26, wherein said plasticizer(s) are water soluble.

31. The pharmaceutical composition of claim 30, wherein said plasticizer(s) are a combination of several water soluble plasticizers.

32. The pharmaceutical composition of claim 26, wherein said plasticizer(s) are a combination of water soluble plasticizers and water insoluble plasticizers.

33. The pharmaceutical composition of claim 26, wherein said plasticizer is triethyl citrate.

34. The pharmaceutical composition of claim 26, wherein said coating further comprises lubricant(s).

35. The pharmaceutical composition of claim 33, wherein the lubricant is talc extra fine.

36. The pharmaceutical composition of claim 26, wherein said core is in tablet form.

37. The pharmaceutical composition of claim 26, wherein the core further comprises at least one disintegrant.

38. The pharmaceutical composition of claim 37, wherein the core comprises between 0.5% and 20% by weight of disintegrant.

39. The pharmaceutical composition of claim 38, wherein said disintegrant comprises pre-gelatinized starch.

40. The pharmaceutical composition of claim 26, having a weight of less than 150 mg.

41. The pharmaceutical composition of claim 26, comprising 1.56 mg of rasagiline mesylate.

42. The pharmaceutical composition of claim 26, comprising 0.78 mg of rasagiline mesylate.

43. The pharmaceutical composition of claim 41, further comprising mannitol, colloidal silicon dioxide, starch NF, pregelatinized starch, stearic acid, talc, hypromellose, methacrylic acid-ethyl acrylate copolymer, talc extra fine, and triethyl citrate.

44. The pharmaceutical composition of claim 41, consisting of 79.84 mg of mannitol, 0.6 mg of colloidal silicon dioxide, 1.56 mg of rasagiline mesylate, 10.0 mg of starch NF, 20.0 mg of pregelatinized starch, 2.0 mg of stearic acid, 2.0 mg of talc, 4.8 mg of hypromellose, 6.25 mg of methacrylic acid-ethyl acrylate copolymer, 1.25 mg of triethyl citrate, and 3.1 mg of talc extra fine.

45. The pharmaceutical composition of claim 42, consisting of 80.62 mg of mannitol, 0.6 mg of colloidal silicon dioxide, 0.78 mg of rasagiline mesylate, 10.0 mg of starch NF, 20.0 mg of pregelatinized starch, 2.0 mg of stearic acid, 2.0 mg of talc, 4.8 mg of hypromellose, 6.25 mg of methacrylic acid-ethyl acrylate copolymer, 1.25 mg of triethyl citrate, and 3.1 mg of talc extra fine.

46. A method of treating a patient suffering from Parkinson's disease comprising administering to the patient a pharmaceutical composition of any one of claims 1 to 45.

47. The method of claim 46, wherein said patient suffers from delayed gastric emptying.
